(19) United States
(12) Patent Application Publication
(10) Pub. No.: US 2015/0005881 A1
(43) Pub. Date: Jan. 1, 2015

(54) Spinal Fusion Implant Enabling Diverse-Angle and Limited-Visibility Insertion

(71) Applicants: Robert A. Connor, Forest Lake, MN (US); Hart Garner, Edina, MN (US)

(72) Inventors: Robert A. Connor, Forest Lake, MN (US); Hart Garner, Edina, MN (US)

(21) Appl. No.: 13/932,695

(22) Filed: Jul. 1, 2013

(51) Int. Cl. A61F 2/44

(52) U.S. Cl. CPC A61F 2/4455
USPC 623/17.16

(57) ABSTRACT

This invention can be embodied as a device implanted into an intervertebral disk space comprising: a distal portion shaped like a rounded rectangular, trapezoidal, or elliptical column; and a proximal portion shaped like a convex, concave, or straight-walled frustum. The proximal portion spans between 25% and 75% of the implant length. This invention can also be a method wherein a recess is drilled into the intervertebral disk tissue and the adjacent vertebrae such that the proximal portion of the implant fits snugly into the recess. This device and method can enable minimally-invasive insertion of the implant from a relatively wide range of entry angles and under conditions of limited visibility. This is especially advantageous for lateral insertion into a lower section of the spine such as the Lumbar 5 Sacral 1 disk space or the Lumbar 4 Lumbar 5 disk space.

### CROSS-REFERENCE TO RELATED APPLICATIONS

Not Applicable

### FEDERALLY SPONSORED RESEARCH

Not Applicable

### SEQUENCE LISTING OR PROGRAM

Not Applicable

### BACKGROUND

### Field of Invention

This invention relates to intervertebral spinal fusion implants.

### INTRODUCTION

This invention relates to intervertebral disk space implants for fusion of adjacent spinal vertebrae. There are many devices and methods for intervertebral disk space implants in the prior art which can promote spinal fusion, but there remain regions of the spine which are particularly challenging to treat with currently-available devices and methods without encountering critical anatomical structures. For example, lateral insertion of intervertebral implants into the lower section of the spine can be particularly challenging, especially for insertion of implants into the Lumbar 5 Sacral 1 (L5-S1) disk space or the Lumbar 4 Lumbar 5 (L4-L5) disk space. Limited visibility is also a challenge for insertion of implants into these lower disk spaces.

The ability to insert an intervertebral disk space implant from a wide range of entry angles can help to meet this need. There is a need for implant devices and methods which guide insertion of intervertebral disk space implants into the intervertebral disk space from a relatively wide range of entry angles and under conditions of limited visibility in order to better avoid critical anatomical structures. This is especially important for lateral insertion of implants into the lower sections of the spine such as the Lumbar 5 Sacral 1 (L5-S1) disk space and the Lumbar 4 Lumbar 5 (L4-L5) disk space. This unmet clinical need is the motivation for this invention.

### Categorization and Review of the Prior Art

Before disclosing this invention, it is useful to first thoroughly review the related prior art. That is what we do in this categorization and review of the prior art. As part of this review, we have categorized the relevant prior art into general categories. With the complexity of this field and the volume of patents therein, seeking to categorize all relevant examples of prior art into discrete categories is challenging. Some examples of prior art span multiple categories and no categorization scheme is perfect. However, even an imperfect categorization scheme can serve a useful purpose for reviewing the prior art.

In the categorization and review of the prior art herein, we have identified and classified over 130 examples of prior art. Writing up individual reviews for each of these 130+ examples would be prohibitively lengthy and would also be less useful for the reader, who would have to wade through these 130+ individual reviews. It is more efficient for the reader to be presented with these 130+ examples of prior art having been grouped into nine general categories, wherein these nine general categories are then reviewed and discussed. To help readers who may wish to dig further into examples within a particular category or to second guess our categorization scheme, we also provide relatively-detailed information on each example of the prior art, including the patent (application) title and date in addition to the inventors and patent (application) number.

The six categories which we use to categorize the 130+ examples of prior art for this review are as follows: (1) generally linear wedge-shaped implants with little (or no) proximal flanges or endplates; (2) generally linear wedge-shaped implants with rotating members; (3) generally linear wedge-shaped implants with modest proximal flanges or endplates; (4) oblong, elliptical, lipstick, or other-convex shaped implants with little (or no) proximal flanges or endplates; (5) threaded or ridged frustal or cylindrical implants with little (or no) proximal flanges or endplates; (6) threaded or ridged frustal or cylindrical implants with modest proximal flanges or endplates; (7) horseshoe, horse hoof, or kidney shaped linear implants with modest proximal flanges or endplates; (8) bulbous implants with proximal flanges or endplates; and (9) intervertebral bone drills with the option of a beveled-end bit.

1. Generally Linear Wedge-Shaped Implants with Little (or No) Proximal Flanges or Endplates

This category of art includes intervertebral implants for spinal vertebrae fusion which have: generally-linear sides with the possible exception of ridges or holes to engage the vertebrae and foster the ingrowth of bone; a generally-trapezoidal vertical longitudinal cross-sectional shape; and little (or no) proximal flange or perpendicular endplate. These implants can have holes through which screws are inserted to further attach the implants to the adjacent vertebrae, but we do not include such screws when analyzing and categorizing the basic shape of the implant. Prior art which appears to be best categorized into this category includes the following U.S. patents: U.S. Pat. No. 5,425,772 (Brantigan, Jun. 20, 1995, “Prosthetic Implant for Intervertebral Spinal Fusion”); U.S. Pat. No. 7,850,736 (Heinz, Dec. 14, 2010, “Vertebral Fusion Implants and Methods of Use”); U.S. Pat. No. 7,972,365 (Michelson, Jul. 5, 2011, “Spinal Implant Having Deployable Bone Engaging Projections and Method for Installation Thereof”); U.S. Pat. No. 8,097,037 (Serhan et al., Jan. 17, 2012, “Methods and Devices for Correcting Spinal Deformities”); U.S. Pat. No. 8,303,601 (Bandeira et al., Nov. 6, 2012, “Collet-Activated Distraction Wedge Inserter”); and U.S. Pat. No. 8,439,977 (Kostuik et al., May 14, 2013, “Spinal Interbody Spacer”).

Prior art which appears to be best categorized into this category also includes the following U.S. patent applications: 20010031254 (Bianchi et al., Oct. 18, 2001, “Assembled Implant”); 20050038511 (Martz et al., Feb. 17, 2005, “Transforaminal Lumbar Interbody Fusion (TLIF) Implant Surgical Procedure and Instruments for Insertion of Spinal Implant in a Spinal Disc Space”); 20080154375 (Serhan et al., Jun. 26, 2008, “Methods and Devices for Correcting Spinal Deformities”); 20080281425 (Thalgott et al., Nov. 13, 2008, “Orthopaedic Implants and Prostheses”); 20090210058 (Barrett, Aug. 20, 2009, “Anterior Lumbar Interbody Graft”); 20090210062 (Thalgott et al., Aug. 20, 2009, “Orthopaedic Implants and Prostheses”); 20090270991 (Michelson, Oct. 29, 2009, “Spinal Fusion Implant with Bone Screws”); 20100268349 (Bianchi et al., Oct. 21, 2010, “Assembled Implant”); 20100305702 (Michelson, Dec. 2, 2010, “Spinal Implant Having Deployable Bone Engaging Projections and Method for Installation Thereof”); 20110082555 (Martz et al., Apr. 7, 2011, “Transforaminal Lumbar Interbody Fusion (TLIF) Implant Surgical Procedure and Instruments for Insertion of Spinal Implant in a Spinal Disc Space”); and 20120158149 (Kostuik et al., Jun. 21, 2012, “Spinal Interbody Spacer”).

2. Generally Linear Wedge-Shaped Implants with Rotating Members

This category of art includes intervertebral implants for spinal vertebrae fusion which have: generally-linear sides with the possible exception of ridges or holes to engage the vertebrae and foster the ingrowth of bone; a generally-trapezoidal vertical longitudinal cross-sectional shape; little (or no) proximal flange or perpendicular endplate; and a rotating member which engages the vertebral ends after implantation. Prior art which appears to be best categorized into this category includes U.S. Pat. No. 7,771,475 (Michelson, Aug. 10, 2010, “Spinal Implant Having Deployable Bone Engaging Projections”) and U.S. Patent Application 20110166655 (Michelson, Jul. 7, 2011, “Spinal Implant Having Deployable Bone Engaging Projections”).

3. Generally Linear Wedge-Shaped Implants with Modest Proximal Flanges or Endplates

This category of art includes intervertebral implants for spinal vertebrae fusion which have: generally-linear sides with the possible exception of ridges or holes to engage the vertebrae and foster the ingrowth of bone; a generally-trapezoidal vertical longitudinal cross-sectional shape; and a modest proximal flange or perpendicular endplate. Proximal endplates tend to join to the longitudinal main body of the implant in a perpendicular manner forming roughly-90-degree angles. Proximal flanges tend to expand outward from the central longitudinal axis of the main body of the implant in an arcuate manner like the distal end of a trumpet. The modest flanges or perpendicular endplates of implants in this category can be useful for securely attaching the implant to the vertebrae with screws or for preventing over-insertion, but they do not have sufficient longitudinal depth nor the proper shape to guide insertion of the implant into the intervertebral space from a wide array of entry angles. These implants can have holes through which screws are inserted to further attach the implants to the adjacent vertebrae, but we do not include such screws when analyzing and categorizing the basic shape of the implant.

Prior art which appears to be best categorized into this category includes the following U.S. patents: U.S. Pat. No. 5,484,437 (Michelson, Jan. 16, 1996, “Apparatus and Method of Inserting Spinal Implants”); U.S. Pat. No. 5,505,732 (Michelson, Apr. 9, 1996, “Apparatus and Method of Inserting Spinal Implants”); U.S. Pat. No. 5,797,909 (Michelson, Aug. 25, 1998, “Apparatus for Inserting Spinal Implants”); U.S. Pat. No. 6,066,175 (Henderson et al., May 23, 2000, “Fusion Stabilization Chamber”); U.S. Pat. No. 6,096,038 (Michelson, Aug. 1, 2000, “Apparatus for Inserting Spinal Implants”); U.S. Pat. No. 6,270,498 (Michelson, Aug. 7, 2001, “Apparatus for Inserting Spinal Implants”); U.S. Pat. No. 6,770,074 (Michelson, Aug. 3, 2004, “Apparatus for Use in Inserting Spinal Implants”); U.S. Pat. No. 6,837,905 (Lieberman, Jan. 4, 2005, “Spinal Vertebral Fusion Implant and Method”); U.S. Pat. No. 6,875,213 (Michelson, Apr. 5, 2005, “Method of Inserting Spinal Implants with the Use of Imaging”); U.S. Pat. No. 7,399,303 (Michelson, Jul. 15, 2008, “Bone Cutting Device and Method for Use Thereof”); U.S. Pat. No. 7,431,722 (Michelson, Oct. 7, 2008, “Apparatus Including a Guard Member Having a Passage with a Non-Circular Cross Section for Providing Protected Access to the Spine”); U.S. Pat. No. 7,993,347 (Michelson, Aug. 9, 2011, “Guard for Use in Performing Human Interbody Spinal Surgery”); U.S. Pat. No. 8,100,955 (Blain et al., Jan. 24, 2012, “Orthopedic Expansion Fastener”); U.S. Pat. No. 8,100,975 (Waugh et al., Jan. 24, 2012, “Intervertebral Implants with Attachable Flanges and Methods of Use”); U.S. Pat. No. 8,114,162 (Bradley, Feb. 14, 2012, “Spinal Fusion Implant and Related Methods”); U.S. Pat. No. 8,425,514 (Anderson et al., Apr. 23, 2013, “Spinal Fixation Device”); and U.S. Pat. No. 8,425,558 (McCormack et al., Apr. 23, 2013, “Vertebral Joint Implants and Delivery Tools”).

Prior art which appears to be best categorized into this category also includes the following U.S. patent applications: 20060235403 (Blain, Oct. 19, 2006, “Flanged Interbody Fusion Device with Locking Plate”); 20060235409 (Blain, Oct. 19, 2006, “Flanged Interbody Fusion Device”); 20060235411 (Blain et al., Oct. 19, 2006, “Orthopedic Expansion Fastener”); 20060235518 (Blain, Oct. 19, 2006, “Flanged Interbody Fusion Device with Fastener Insert and Retaining Ring”); 20060235533 (Blain, Oct. 19, 2006, “Flanged Interbody Fusion Device with Hinge”); 20070055252 (Blain et al., Mar. 8, 2007, “Flanged Interbody Fusion Device with Oblong Fastener Apertures”); 20100274358 (Mueller et al., Oct. 28, 2010, “Spine Stabilization Device and Method and Kit for Its Implantation”); 20110046682 (Stephan et al., Feb. 24, 2011, “Expandable Fixation Assemblies”); 20120041559 (Melkent et al., Feb. 16, 2012, “Interbody Spinal Implants with Extravertebral Support Plates”); 20120158056 (Blain, Jun. 21, 2012, “Orthopedic Expansion Fastener”); and 20120191198 (Link et al., Jul. 26, 2012, “Cervical Intervertebral Prosthesis”).

4. Oblong, Elliptical, Lipstick, or Other-Convex Shaped Implants with Little (or No) Proximal Flanges or Endplates

This category of art includes intervertebral implants for spinal vertebrae fusion which have: a vertical longitudinal cross-sectional shape which is generally oblong, elliptical, lipstick-shaped, or other arcuate-convex shape; and little (or no) proximal flange or perpendicular endplate. These implants can have holes through which screws are inserted to further attach the implants to the adjacent vertebrae, but we do not include such screws when analyzing and categorizing the basic shape of the implant.

Prior art which appears to be best categorized into this category includes the following U.S. patents: U.S. Pat. No. 5,306,307 (Senter et al., Apr. 26, 1994, “Spinal Disk Implant”); U.S. Pat. No. 6,277,149 (Boyle et al., Aug. 21, 2001, “Ramp-Shaped Intervertebral Implant”); U.S. Pat. No. 6,530,955 (Boyle et al., Mar. 11, 2003, “Ramp-Shaped Intervertebral Implant”); U.S. Pat. No. 7,749,269 (Peterman et al., Jul. 6, 2010, “Spinal System and Method Including Lateral Approach”); U.S. Pat. No. 7,776,095 (Peterman et al., Aug. 17, 2010, “Spinal System and Method Including Lateral Approach”); U.S. Pat. No. 7,988,734 (Peterman et al., Aug. 2, 2011, “Spinal System and Method Including Lateral Approach”); and U.S. Pat. No. 8,460,380 (Copf et al., Jun. 11, 2013, “Intervertebral Implant and Surgical Method for Spondylodesis of a Lumbar Vertebral Column”).

Prior art which appears to be best categorized into this category also includes the following U.S. patent applications: 20060217806 (Peterman et al., Sep. 28, 2006, “Spinal System and Method Including Lateral Approach”); 20070260320 (Peterman et al., Nov. 8, 2007, “Spinal System and Method Including Lateral Approach”); 20100262249 (Peterman et al., Oct. 14, 2010, “Spinal System and Method Including Lateral Approach”); 20110251689 (Seifert et al., Oct. 13, 2011, “Intervertebral Implant”); 20110295372 (Peterman et al., Dec. 1, 2011, “Spinal System and Method Including Lateral Approach”); and 20120330417 (Zipnick, Dec. 27, 2012, “Tapered Arcuate Intervertebral Implant”).

5. Threaded or Ridged Frustal or Cylindrical Implants with Little (or No) Proximal Flanges or Endplates

This category of art includes intervertebral implants for spinal vertebrae fusion which are generally threaded or ridged cylinders or frustums and have little (or no) proximal flange or perpendicular endplate. Cylindrical or frustal implants with spiral threads can be inserted into the intervertebral space by engaging rotation, in a manner similar to the way in which screws are inserted into a solid by rotation. Cylindrical or frustal implants with proximally-angled ridges can be inserted into the intervertebral space by tapping and the ridges can engage the vertebral ends to keep the implant from coming out. These implants can have holes through which screws are inserted to further attach the implants to the adjacent vertebrae, but we do not include such screws when analyzing and categorizing the basic shape of the implant.

Prior art which appears to be best categorized into this category includes the following U.S. patents: U.S. Pat. No. 6,063,088 (Winslow, May 16, 2000, “Method and Instrumentation for Implant Insertion”); U.S. Pat. No. 6,210,412 (Michelson, Apr. 3, 2001, “Method for Inserting Frusto-Conical Interbody Spinal Fusion Implants”); U.S. Pat. No. 6,436,098 (Michelson, Aug. 20, 2002, “Method for Inserting Spinal Implants and for Securing a Guard to the Spine”); U.S. Pat. No. 6,923,810 (Michelson, Aug. 2, 2005, “Frusto-Conical Interbody Spinal Fusion Implants”); U.S. Pat. No. 7,291,149 (Michelson, Nov. 6, 2007, “Method for Inserting Interbody Spinal Fusion Implants”); U.S. Pat. No. 7,452,359 (Michelson, Nov. 18, 2008, “Apparatus for Inserting Spinal Implants”); U.S. Pat. No. 7,534,254 (Michelson, May 19, 2009, “Threaded Frusto-Conical Interbody Spinal Fusion Implants”); U.S. Pat. No. 7,662,185 (Alfaro et al., Feb. 16, 2010, “Intervertebral Implants”); U.S. Pat. No. 7,691,148 (Michelson, Apr. 6, 2010, “Frusto-Conical Spinal Implant”); U.S. Pat. No. 7,828,800 (Michelson, Nov. 9, 2010, “Threaded Frusto-Conical Interbody Spinal Fusion Implants”); U.S. Pat. No. 7,942,933 (Michelson, May 17, 2011, “Frusto-Conical Spinal Implant”); U.S. Pat. No. 8,057,475 (Michelson, Nov. 15, 2011, “Threaded Interbody Spinal Fusion Implant”); U.S. Pat. No. 8,226,652 (Michelson, Jul. 24, 2012, “Threaded Frusto-Conical Spinal Implants”); and U.S. Pat. No. 8,409,292 (Michelson, Apr. 2, 2013, “Spinal Fusion Implant”).

Prior art which appears to be best categorized into this category also includes the following U.S. patent applications: 20010032017 (Alfaro et al., Oct. 18, 2001, “Intervertebral Implants”); 20030036798 (Alfaro et al., Feb. 20, 2003, “Intervertebral Implants”); 20040044409 (Alfaro et al., Mar. 4, 2004, “Intervertebral Implants”); 20090228107 (Michelson, Sep. 10, 2009, “Threaded Frusto-Conical Interbody Spinal Fusion Implants”); 20100217394 (Michelson, Aug. 26, 2010, “Frusto-Conical Spinal Implant”); 20110054529 (Michelson, Mar. 3, 2011, “Threaded Interbody Spinal Fusion Implant”); 20120053695 (Michelson, Mar. 1, 2012, “Threaded Frusto-Conical Spinal Implants”); and 20120290092 (Michelson, Nov. 15, 2012, “Spinal Implants”).

6. Threaded or Ridged Frustal or Cylindrical Implants with Modest Proximal Flanges or Endplates

This category of art includes intervertebral implants for spinal vertebrae fusion which are generally threaded or ridged cylinders or frustums and have a modest proximal flange or perpendicular endplate. Cylindrical or frustal implants with spiral threads can be inserted into the intervertebral space by engaging rotation, in a manner similar to the way in which screws are inserted into a solid by rotation. Cylindrical or frustal implants with proximally-angled ridges can be inserted into the intervertebral space by tapping and the ridges can engage the vertebral ends to keep the implant from coming out. Proximal endplates tend to join to the longitudinal main body of the implant in a perpendicular manner forming roughly-90-degree angles. Proximal flanges tend to expand outward from the central longitudinal axis of the main body of the implant in an arcuate manner like the distal end of a trumpet. The modest flanges or perpendicular plates of implants in this category can be useful for securely attaching the implant to the vertebrae with screws or for preventing over-insertion, but do not have sufficient longitudinal depth nor the proper shape to guide insertion of the implant into the intervertebral space from a wide array of entry angles. These implants can have holes through which screws are inserted to further attach the implants to the adjacent vertebrae, but we do not include such screws when analyzing and categorizing the basic shape of the implant. Prior art which appears to be best categorized into this category includes U.S. patents: U.S. Pat. No. 6,926,737 (Jackson, Aug. 9, 2005, “Spinal Fusion Apparatus and Method”) and U.S. Pat. No. 8,328,555 (Engman, Dec. 11, 2012, “Implant”). Prior art which appears to be best categorized into this category also includes U.S. Patent Application 20020116065 (Jackson, Aug. 22, 2002, “Spinal Fusion Apparatus and Method”).

7. Horseshoe, Horse Hoof, or Kidney Shaped Linear Implants with Modest Proximal Flanges or Endplates

This category of art includes intervertebral implants for spinal vertebrae fusion with a horizontal cross-section which is generally shaped like a horseshoe, horse hoof, or kidney and have a modest proximal flange or perpendicular endplate. Proximal endplates tend to join to the longitudinal main body of the implant in a perpendicular manner forming roughly-90-degree angles. Proximal flanges tend to expand outward from the central longitudinal axis of the main body of the implant in an arcuate manner like the distal end of a trumpet. The modest flanges or perpendicular plates of implants in this category can be useful for securely attaching the implant to the vertebrae with screws or for preventing over-insertion, but do not have sufficient longitudinal depth nor the proper shape to guide insertion of the implant into the intervertebral space from a wide array of entry angles. These implants can have holes through which screws are inserted to further attach the implants to the adjacent vertebrae, but we do not include such screws when analyzing and categorizing the basic shape of the implant.

Prior art which appears to be best categorized into this category includes the following U.S. patents: U.S. Pat. No. 6,730,127 (Michelson, May 4, 2004, “Flanged Interbody Spinal Fusion Implants”); U.S. Pat. No. 7,163,561 (Michelson, Jan. 16, 2007, “Flanged Interbody Spinal Fusion Implants”); U.S. Pat. No. 7,794,502 (Michelson, Sep. 14, 2010, “Implant with Openings Adapted to Receive Bone Screws”); U.S. Pat. No. 7,935,149 (Michelson, May 3, 2011, “Spinal Fusion Implant with Bone Screws”); U.S. Pat. No. 8,167,946 (Michelson, May 1, 2012, “Implant with Openings Adapted to Receive Bone Screws”); U.S. Pat. No. 8,323,343 (Michelson, Dec. 4, 2012, “Flanged Interbody Spinal Fusion Implants”); U.S. Pat. No. 8,328,872 (Duffield et al., Dec. 11, 2012, “Intervertebral Fusion Implant”); and U.S. Pat. No. 8,353,959 (Michelson, Jan. 15, 2013, “Push-In Interbody Spinal Fusion Implants for Use with Self-Locking Screws”).

Prior art which appears to be best categorized into this category also includes the following U.S. patent applications: 20070106388 (Michelson, May 10, 2007, “Flanged Interbody Spinal Fusion Implants”); 20090062921 (Michelson, Mar. 5, 2009, “Implant with Openings Adapted to Receive Bone Screws”); 20100057206 (Duffield et al., Mar. 4, 2010, “Intervertebral Fusion Implant”); 20100312345 (Duffield et al., Dec. 9, 2010, “Intervertebral Fusion Implant”); 20120078373 (Gamache et al., Mar. 29, 2012, “Stand Alone Intervertebral Fusion Device”); 20120130495 (Duffield et al., May 24, 2012, “Intervertebral Fusion Implant”); 20120130496 (Duffield et al., May 24, 2012, “Intervertebral Fusion Implant”); 20120179259 (McDonough et al., Apr. 12, 2012, “Intervertebral Implants, Systems, and Methods of Use”); 20120283838 (Rhoda, Nov. 8, 2012, “Intervertebral Implant”); 20130060339 (Duffield et al., Mar. 7, 2013, “Intervertebral Fusion Implant”); 20130085573 (Lemoine et al., Apr. 4, 2013, “Interbody Vertebral Spacer”); and 20130096688 (Michelson, Apr. 18, 2013, “Interbody Spinal Fusion Implant Having a Trailing End with at Least One Stabilization Element”).

8. Bulbous Implants with Proximal Flanges or Endplates

This category of art includes intervertebral implants for spinal vertebrae fusion with a horizontal cross-section which includes a bulbous distal portion and a modest proximal flange or perpendicular endplate. Some implants in this category have a vertical longitudinal cross-sectional shape which is similar to that of a stylized goldfish or the end of a plumb bob. Proximal endplates tend to join to the longitudinal main body of the implant in a perpendicular manner forming roughly-90-degree angles. Proximal flanges tend to expand outward from the central longitudinal axis of the main body of the implant in an arcuate manner like the distal end of a trumpet. The modest flanges or perpendicular plates of implants in this category can be useful for securely attaching the implant to the vertebrae with screws or for preventing over-insertion, but do not have sufficient longitudinal depth nor the proper shape to guide insertion of the implant into the intervertebral space from a wide array of entry angles. These implants can have holes through which screws are inserted to further attach the implants to the adjacent vertebrae, but we do not include such screws when analyzing and categorizing the basic shape of the implant.

Prior art which appears to be best categorized into this category includes U.S. Pat. No. 7,963,991 (Conner et al., Jun. 21, 2011, “Spinal Implants and Methods of Providing Dynamic Stability to the Spine”). Prior art which appears to be best categorized into this category also includes the following U.S. patent applications: 20090138015 (Conner et al., May 28, 2009, “Spinal Implants and Methods”); 20090138084 (Conner et al., May 28, 2009, “Spinal Implants and Methods”); 20090149959 (Conner et al., Jun. 11, 2009, “Spinal Implants and Methods”); 20090149959 (Conner et al., Jul. 11, 2009, “Spinal Implants and Methods”); 20090171461 (Conner et al., Jul. 2, 2009, “Spinal Implants and Methods”); 20090171461 (Conner et al., Jul. 2, 2009, “Spinal Implants and Methods”); 20090270989 (Conner et al., Oct. 29, 2009, “Spinal Implants and Methods”); and 20090270989 (Conner et al., Oct. 29, 2009, “Spinal Implants and Methods”).

9. Intervertebral Bone Drills with the Option of a Beveled-End Bit

This category of art focuses more on the tools and methods for the insertion of intervertebral implants for fusion than on the shapes of the implants themselves. In particular, this category includes drills for removing vertebral bone and/or intervertebral disk tissue in preparation for insertion of fusion-inducing implants. There are a large number of drills and related tools to assist in the insertion of intervertebral implants. For the purposes of this categorization, we have included bone drills in this category that appear to include the option of a beveled-end bit that is capable of creating a convex recess in the vertebral bone ends and intervertebral space that could accommodate an implant with a flanged proximal section.

Prior art which appears to be best categorized into this category includes the following U.S. patents: U.S. Pat. No. 5,489,307 (Kuslich et al., Feb. 6, 1996, “Spinal Stabilization Surgical Method”); U.S. Pat. No. 5,720,748 (Kuslich et al., Feb. 24, 1998, “Spinal Stabilization Surgical Apparatus”); U.S. Pat. No. 5,928,242 (Kuslich et al., Jul. 27, 1999, “Laparoscopic Spinal Stabilization Method”); U.S. Pat. No. 5,947,971 (Kuslich et al., Sep. 7, 1999, “Spinal Stabilization Surgical Apparatus”); U.S. Pat. No. 6,080,155 (Michelson, Jun. 27, 2000, “Method of Inserting and Preloading Spinal Implants”); U.S. Pat. No. 6,447,512 (Landry et al., Sep. 10, 2002, “Instrument and Method for Implanting an Interbody Fusion Device”); U.S. Pat. No. 6,524,312 (Landry et al., Feb. 25, 2003, “Instrument and Method for Implanting an Interbody Fusion Device”); U.S. Pat. No. 6,616,671 (Landry et al., Sep. 9, 2003, “Instrument and Method for Implanting an Interbody Fusion Device”); U.S. Pat. No. 7,207,991 (Michelson, Apr. 24, 2007, “Method for the Endoscopic Correction of Spinal Disease”); and U.S. Pat. No. 8,251,997 (Michelson, Aug. 28, 2012, “Method for Inserting an Artificial Implant Between Two Adjacent Vertebrae Along a Coronal Plane”).

Prior art which appears to be best categorized into this category also includes the following U.S. patent applications: 20080255564 (Michelson, Oct. 16, 2008, “Bone Cutting Device”); 20110264225 (Michelson, Oct. 27, 2011, “Apparatus and Method for Creating an Implantation Space in a Spine”); 20120071984 (Michelson, Mar. 22, 2012, “Method for Inserting an Artificial Implant Between Two Adjacent Vertebrae Along a Coronal Plane”); 20120271312 (Jansen, Oct. 25, 2012, “Spline Oriented Indexing Guide”); and 20120323331 (Michelson, Dec. 20, 2012, “Spinal Implant and Instruments”.

### SUMMARY AND ADVANTAGES OF THIS INVENTION

This invention is a device and method for fusing spinal vertebrae. This invention can be embodied in a device that is implanted into the intervertebral disk space between two adjacent spinal vertebrae. Apart from optional repeated protrusions, repeated ridges, holes, or independently-movable fastening members such as screws, the basic shape of this implant includes: (a) a distal portion that is generally shaped like a rounded rectangular, trapezoidal, or elliptical column; and (b) a proximal portion that is generally shaped like a convex, concave, or straight-walled frustum. The proximal portion of the implant spans between 25% and 75% of the length of the implant. The distal portion spans the remaining length of the implant.

This invention can be also be embodied in method for implantation of such a device wherein a relatively deep and convex recess is drilled into the intervertebral disk space tissue and adjacent vertebral ends such that: the proximal portion of such an implant fits relatively snugly into the recess when implanted; and proximal end of the implant fits relatively flush with the pre-drilling lateral wall of the vertebrae when the implant is implanted.

This invention provides advantages over devices and methods for spinal fusion in the prior art, especially for lateral insertion of an intervertebral implant into a lower section of the spine such as the Lumbar 5 Sacral 1 (L5-S1) disk space or the Lumbar 4 Lumbar 5 (L4-L5) disk space. For example, drilling a frustum-shaped recess into the vertebrae (contiguous to the intervertebral disk space) can help to guide insertion of the spinal fusion implant into the intervertebral disk space from a relatively wide range of entry angles. This can be very advantageous for avoiding critical anatomical structures (such as nerves, muscles, and blood vessels) when laterally inserting a spinal fusion implant into a lower section of the spine such as the Lumbar 5 Sacral 1 (L5-S1) disk space or the Lumbar 4 Lumbar 5 (L4-L5) disk space.

Also, there is limited direct visibility for insertion of implants into the lower section of the spine including the Lumbar 1 Sacral 1 disk space and Lumbar 4 Lumbar 5 disk space. It is difficult to insert implants in the prior art into these areas in a minimally invasive manner. The frustum-shaped bone recess of the invention disclosed herein combined with the shape of the implant itself solves this problem and enables minimally-invasive insertion of a spinal fusion implant into these lower disk spaces under conditions of limited direct visibility.

The invention disclosed herein also offers biomechanical advantages in cases wherein the intervertebral disk space should be expanded to correct shrinkage which has occurred due to disk pathology. The implant disclosed herein applies expanding force to the vertebral bone ends over a relatively broad contact area. Application of expanding force through a broader contact area can decrease the chances of vertebral bone fracture during insertion.

Finally, designing geometric complementarity between the shape of a drilled recess and the proximal portion of the implant can ensure that the implant will fit relatively flush with the spinal column after implantation. Although the prior art includes spinal implants with modest proximal flanges and endplates that attach to the lateral exterior of vertebrae after implantation, the prior art does not appear to disclose a device with a proximal portion of sufficient size and the proper shape to offer such guidance for diverse-angle and limited-visibility insertion of spinal implants.

### INTRODUCTION TO THE FIGURES

FIGS. 1 through 19 show examples of how this invention can be embodied but they do not limit the full generalizability of the claims.

FIGS. 1 through 3 show a three-view stylized (graphically simplified) sequence of one example of how this invention can be embodied an implant that is inserted between two adjacent spinal vertebrae. These figures help to show the anatomical context within which this implant is used.

FIG. 4 defines a geometric axial framework for the implant (including longitudinal, vertical, and horizontal axes) that enables precise specification of the implant shape.

FIG. 5 shows how the intervertebral implant can be conceptually and longitudinally divided into four segments for precise specification of the implant shape.

FIGS. 6 and 7 show cross-sectional views of an example of how this invention can be embodied in an implant with a distal portion that is shaped like a rectangular column with rounded edges and a proximal portion that is shaped like a section of a convex cone.

FIGS. 8 and 9 show cross-sectional views of two examples of how this invention can be embodied in an implant with a distal portion that is shaped like a rectangular column with ridges and rounded edges, a proximal portion that is shaped like a section of a concave cone, and two different lengths of the distal portion.

FIGS. 10 and 11 show cross-sectional views of two examples of how this invention can be embodied in an implant with a distal portion that is shaped like a rectangular column with ridges and rounded edges, a proximal portion that is shaped like a section of a convex cone, and two different lengths of the distal portion.

FIGS. 12 and 13 show cross-sectional views of two examples of how this invention can be embodied in an implant with a distal portion that is shaped like a rectangular column with ridges and rounded edges, a proximal portion that is shaped like a section of a straight-walled cone, and two different lengths of the distal portion.

FIGS. 14 through 16 show three views of an example of how this invention can be embodied in an implant comprising: a distal portion shaped like a rectangular column with rounded edges, ridges on its upper and lower surfaces, and holes; and a proximal portion shaped like a section of a cone with an elliptical base and straight walls from its base to its peak and a hole.

FIGS. 17 through 19 show three views of an example of how this invention can be embodied in an implant comprising: a distal portion shaped like a trapezoid column (for lordotic applications) with rounded edges, ridges on its upper and lower surfaces, and holes; and a proximal portion shaped like a section of a cone with an elliptical base and straight walls from its base to its peak and a hole.

### DETAILED DESCRIPTION OF THE FIGURES

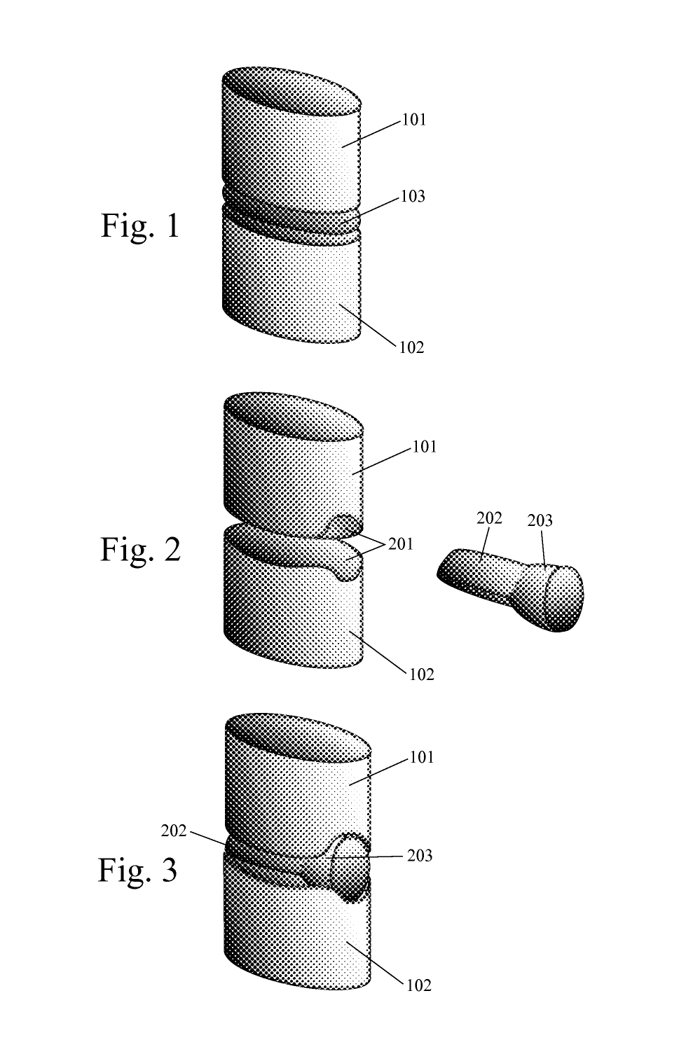

FIGS. 1 through 19 show various examples of how this invention can be embodied in devices and methods for promoting fusion of spinal vertebrae, but they do not limit the full generalizability of the claims.

FIGS. 1 through 3 show three sequential oblique-angle views of an embodiment of this invention in an intervertebral implant that is inserted into the disk space between two adjacent spinal vertebrae. This three-view sequence is particularly useful for seeing the anatomical context within which this implant is used.

In FIG. 1, two adjacent spinal vertebrae, 101 and 102, are graphically represented by simple elliptical cylinders and the intervertebral disk, 103, that is between them is graphically represented by a simple elliptical disk. The graphic simplicity of these representations instead of using anatomically-correct representations of the vertebrae and disk provides the viewer with a clearer view of how the implant is used. In particular, using graphically-simplified versions of the vertebrae and disk provides a clearer view of the geometry of a recess that is drilled into the vertebrae prior to insertion and how the implant is inserted into the intervertebral space.

FIG. 1 shows the two spinal vertebrae, upper vertebra 101 and lower vertebra 102, prior to drilling and prior to insertion of the intervertebral implant. This helps to show the anatomical context within which the intervertebral implant is used.

FIG. 2 shows these same vertebrae, 101 and 102, after the tissue of intervertebral disk 103 has been removed and a frustum-shaped recess 201 has been drilled into the vertebrae prior to insertion of the implant. In this example, recess 201 is drilled into the lateral faces of the vertebrae to prepare for insertion of the implant through the lateral side of the intervertebral disk space. Recess 201 is formed by drilling away an arcuate portion of upper vertebra 101 that is contiguous to the intervertebral disk space, drilling away an arcuate portion of lower vertebra 102 that is contiguous to intervertebral space, and removing tissue from the intervertebral disk between these upper and lower arcuate portions.

Recess 201 can help to guide the insertion of the intervertebral implant into the intervertebral space from a variety of insertion angles. This can be very useful for insertion of an implant into lower sections of the spine (such as the Lumbar 5 Sacral 1 disk space or the Lumbar 4 Lumbar 5 disk space) wherein insertion from a straight-line angle is sometimes infeasible. Recess 201 can also help to ensure that the implant is inserted to the proper depth such that the implant is flush with the pre-drilling lateral sides of the vertebrae. In an example, the walls of recess 201 can receive a proximal portion of the intervertebral implant and prevent either over-insertion or under-insertion of the implant.

In an example, recess 201 can be shaped like a section of a cone (i.e. a frustum). In an example, the cone can be a conventional cone with straight-line walls from the base of the cone to the peak of the cone. In alternative examples, the cone can have convex or concave walls. In an example, the recess can be wider at its proximal portion (closest to the operator) and narrower at its distal portion (furthest into the vertebra). In an example, recess 201 can be shaped like a section of a sphere (e.g. a hemisphere). In an example, recess 201 can be shaped like a section of a rotated polygon.

In an example, this invention can be embodied in a method for fusing spinal vertebrae. In an example, the first step of this method can comprise drilling a recess into a section of the spine comprising two adjacent spinal vertebrae, wherein this recess includes a portion of the intervertebral disk space, a portion of the upper vertebrae that is contiguous the intervertebral disk space, and a portion of the lower vertebrae that is contiguous the intervertebral disk space. In an example, this recess can extend between 25% and 75% of the lateral span of the intervertebral disk space. In an example, this recess can be shaped like a section of a cone or rotated polygon. In an example, this recess can have a wider proximal cross-section than distal cross-section.

In an example, recess 201 can be drilled into vertebrae 101 and 102 using a rotating drill and the resulting bony tissue can be suctioned out through a catheter. In an example, the drill bit can have a shape that is selected from the group consisting of: cone or conic section; section of a sphere; symmetric rotated polygon; and spiral or helix around a cylindrical core. In an example, recess 201 can be drilled before the remaining tissue of the intervertebral disk is removed. In an alternative example, the tissue of the intervertebral disk can be removed before recess 201 is drilled.

The right portion of FIG. 2 also introduces one possible embodiment of the intervertebral implant that is to be inserted into the intervertebral disk space to help fuse the vertebrae together. In an example, the geometric definitions and limitations that we discuss to specify the invention apply to the main body of this implant, excluding any screws or other any fastening members which can be rotated and/or inserted inwards independently of the implant. For example, when we specify the shape of the implant, we are referring to the main body of the implant apart from any screws or other fastening members which may be inserted through the implant to fasten it to the vertebrae.

As shown in FIG. 2, this invention can be embodied in an implant that has two longitudinal portions. This implant has a distal portion 202 that is first inserted into the intervertebral space and a proximal portion 203 that is last inserted into the intervertebral space. In an example, this implant can have a distal-to-proximal longitudinal axis.

In the example that is shown in FIG. 2, the distal portion 202 of the implant spans approximately two-thirds of the distal-to-proximal length of the implant and the proximal portion 203 of the implant spans the remaining one-third of this length. In an example, a distal portion of the implant can span at least 25% and no more than 75% of the distal-to-proximal length of the intervertebral implant. In an example, a proximal portion of the implant can span at least 25% and no more than 75% of the distal-to-proximal length of the intervertebral implant. In an example, the distal portion and the proximal portion together can span all of the distal-to-proximal length of the implant.

In an example, the distal portion 202 can span between 25% and 50% of the distal-to-proximal length of the implant and the proximal portion 203 can span the remaining portion of the distal-to-proximal length of the implant. In an example, the distal portion 202 can span between 50% and 75% of the distal-to-proximal length of the implant and the proximal portion 203 can span the remaining portion of the distal-to-proximal length of the implant.

In an example, the distal portion 202 of the implant that is first inserted into the intervertebral disk space can comprise: a rounded distal end, two lateral surfaces, an upper surface, and a lower surface. In an example, the upper and lower surfaces of the distal portion 202 can be flat and/or smooth. In an example, the upper and lower surfaces of the distal portion can have multiple ridges or other protrusions to prevent the implant from sliding out during implantation, to better grip the vertebrae after implantation, and/or to foster the growth of bone from the vertebrae into the implant after implantation. In an example, the upper and lower surfaces of the distal portion can have multiple holes to foster the growth of bone from the vertebrae into the implant after implantation. In an example, bone can grow completely through these holes to better connect and fuse the vertebrae to each other.

In an example, the upper and lower surfaces of the distal portion can be generally flat apart from a sequence of repeating ridges, protrusions, or holes. In an example, a sequence of repeating ridges or protrusions can have a cross-sectional profile that comprises a sinusoidal wave with variation around a substantially straight line. In an example, a sequence of repeating ridges or protrusions can have a cross-sectional profile that comprises a saw-tooth wave with variation around a substantially straight line. In an example, a sequence of repeating ridges or protrusions can have a cross-sectional profile that comprises a series of peaks above a substantially straight line.

In an example, a best-fitting straight line can be defined for the upper perimeter of a selected longitudinal cross-sectional area of the proximal portion of the implant. In an example, a best-fitting straight line can also be defined for the lower perimeter of this longitudinal cross-section of the proximal portion of the implant. In an example, a best-fitting straight line for a perimeter can be defined as the straight line that minimizes the sum of squared deviations from points along the perimeter. In an example, a best-fitting straight line for a perimeter can be defined as the straight line that minimizes the sum of absolute values of deviations from points along the perimeter. In an example, a best-fitting straight line for a perimeter can be defined as the straight line that remains if one were to geometrically subtract or cancel a repeating wave sequence of ridges, protrusions, or holes from that perimeter.

In an example, a selected longitudinal cross-sectional area can be the longitudinal cross-sectional area with the greatest vertical distance between the lower surface and the upper surface. In an example, a selected longitudinal cross-sectional area can be the longitudinal cross-sectional area that is centrally located between the lateral sides.

In an example, a best-fitting flat plane can be defined for the entire upper surface of the distal portion of the implant. In an example, a best-fitting flat plane can also be defined for the entire lower surface of the distal portion of the implant. In an example, a best-fitting flat plane for a surface can be defined as the flat plane that minimizes the sum of squared deviations from the points on that surface. In an example, a best-fitting flat plane for a surface can be defined as the flat plane that minimizes the sum of absolute values of deviations from the points on that surface. In an example, a best-fitting flat plane for a surface can be defined as the flat plane that remains if one were to geometrically subtract or cancel a repeating sequence of ridges, protrusions, or holes from that surface.

In this example, the best-fitting flat plane for the upper surface of the distal portion 202 of the implant is substantially parallel to the best-fitting flat plane for the lower surface of the distal portion 202 of the implant. In this example, the distal portion of the implant is shaped substantially like a rectangular column, albeit with slightly rounded edges. In this example, the distal portion 202 of the implant is wider (distance between the two lateral surfaces) than it is high (distance between the lower and upper surfaces).

In this example, the upper and lower surfaces of the distal portion 202 are relatively flat and smooth. In an example, the upper and lower surfaces of the distal portion can have a sequence of ridges or other protrusions to engage the vertebrae during and after insertion into the intervertebral disk space. In an example, such ridges or protrusions can foster attachment of the implant to the vertebrae. In an example, the upper and lower surfaces of the distal portion 202 of the implant can have holes. In an example, such holes can foster bone ingrowth and fusion of the upper 101 and lower 102 vertebrae. In an example, bone can grow completely through these holes to better connect and fuse vertebrae 101 and 102 to each other.

In an example, a distal portion of the implant can be shaped substantially like a rectangular column with substantially parallel upper and lower surfaces, with the exception of having rounded edges and a plurality of ridges or other protrusions on its upper and lower surfaces. In an alternative example, the distal portion of the implant can be shaped substantially like an elliptical column with a plurality of ridges or other protrusions on its upper and lower surfaces.

FIG. 2 also shows that the intervertebral implant has a proximal portion 203. This is the portion of the implant which is closest to the operator and last inserted into the intervertebral disk space. In this example, the proximal portion 203 of the implant is shaped substantially like a conic section (e.g. a frustum). In this example, the cone has a circular base.

In an example, the proximal portion 203 of the implant can be shaped substantially like a section of a cone that has a circular base and convex sides from the base to the peak. In an example, the proximal portion 203 of the implant can be shaped substantially like a section of a cone that has a circular base and concave sides from the base to the peak.

In an example, the optimality of having a proximal portion 203 with straight, convex, or concave sides can depend on the range of insertion angles which is possible given the anatomical structures surrounding the segment of the spine which is to be fused. For example, convex sides may be optimal for guiding insertion of the implant to avoid damaging nerves or other organelles from a particular insertion angle. For example, concave sides may be optimal for guiding insertion of the implant to avoid damaging nerves or other organelles from a different insertion angle. In an example, different degrees of proximal portion convexity or concavity can be optimal for different insertion angles and/or for vertebral segments in different locations along the length of the spinal column.

In another example, the proximal portion 203 of the implant can be shaped substantially like a section of a cone that has a elliptical base and straight sides from the base to the peak. In an example, the proximal portion of the implant can be shaped substantially like a section of a cone that has an elliptical base and convex sides from the base to the peak. In an example, the proximal portion of the implant can be shaped substantially like a section of a cone that has an elliptical base and concave sides from the base to the peak. In an example, a shape that is a section of an elliptical cone can be preferred to a shape that is a section of a circular cone in order to better match a distal portion 202 with a greater width than height. In an example, proximal portion 203 can be shaped substantially like a section of a sphere.

In an example, the proximal portion 203 of an implant that is last inserted into the intervertebral disk space can have an uppermost perimeter and a lowermost perimeter. In an example, the best-fitting straight line for the uppermost perimeter of the proximal portion of the implant and the best-fitting straight line for the lowermost perimeter of the proximal portion of the implant can diverge (move apart) as one moves in a distal-to-proximal direction along the proximal portion. In an example, the best-fitting straight line for the uppermost perimeter of the proximal portion and the best-fitting straight line for the lowermost perimeter of the proximal portion can be further apart at the proximal end of the proximal portion than they are at the distal end of the proximal portion. This is the case in the frustum-shaped proximal portion 203 that is shown in FIG. 2.

In an example, the proximal portion 203 of an implant that is last inserted into the intervertebral disk space can comprise an upper surface and a lower surface. In an example, the best-fitting flat plane for the upper surface of the proximal portion of the implant and the best-fitting flat plane for the lower surface of the proximal portion of the implant can diverge (move apart) as one moves in a distal-to-proximal direction along the proximal portion. In an example, the best-fitting flat plane for the upper surface of the proximal portion and the best-fitting flat plane for the lower surface of the proximal portion can be further apart at the proximal end of the proximal portion than they are at the distal end of the proximal portion. This is the case in the frustum-shaped proximal portion 203 that is shown in FIG. 2.

The frustum-shaped proximal portion 203 of the implant that is shown in FIG. 2 is generally arcuate. However, in an example, a proximal portion of an implant can be a polygonal configuration comprised of multiple flat lines and/or flat planes. In an example, a proximal portion of an implant can be shaped like a rotated polygon or a section of a rotated polygon.

In this example, the surfaces of the proximal portion 203 of the implant are substantially smooth. In an alternative example, there can be a plurality of ridges or other protrusions in these surfaces to promote bone ingrowth and/or attachment of the implant to the vertebrae. In an example, there can be one or more holes these surfaces to promote bone ingrowth. In an example, bone can grow completely through these holes to better connect and fuse the vertebrae to each other.

In an example, the distal portion 202 and proximal portion 203 of the intervertebral implant shown in FIG. 2 can be made from one or more materials selected from the group consisting of: metal; polymer; ceramic material; natural bone tissue; and artificial bone tissue. In an example, one or more biologically active agents can be added to foster bone growth and/or attachment of the implant to the vertebrae. In an example, the distal portion 202 and proximal 203 portions can be made of the same materials and/or have a common coating. In an alternative example, the distal portion 202 and proximal 203 portions can be made of different materials and/or have different coatings.

FIG. 2 showed the sequence of two spinal vertebrae, 101 and 102, as well as intervertebral implant (comprising distal portion 202 and proximal portion 203) after recess 201 has been drilled, but before the implant has been inserted into the intervertebral disk space. FIG. 3 now shows these same spinal vertebrae after the implant has been fully inserted into the intervertebral disk space.

As shown in FIG. 3, after the implant has been inserted, the proximal end of the proximal portion 203 of the implant is now within recess 201 and its proximal end is now substantially flush with the pre-drilled lateral surfaces of vertebrae 101 and 102. In an example, having the proximal portion be substantially flush can be defined as the proximal end of the implant being no more than a selected distance away from the pre-drilled lateral surfaces of the vertebrae. In an example, having the proximal portion be substantially flush can be defined as: having the proximal end of the implant be inserted such that is no more than a selected distance interior to the pre-drilled lateral surfaces of the vertebrae; and/or having the proximal end of the implant be inserted such that it extends no more than a selected distance out from the pre-drilled lateral surfaces of the vertebrae. In an example, this selected distance can be 1 mm. In alternative examples, this selected distance can be 5 mm, 10 mm, or 50 mm.

In the example shown in FIG. 3, after insertion, the upper surface of distal portion 202 of the implant is in close and engaging contact with the lower surface of upper vertebrae 101 that is contiguous with the intervertebral disk space and the lower surface of distal portion 202 of the implant is in close and engaging contact with the upper surface of lower vertebrae 102 that is contiguous with the intervertebral disk space. As also shown in FIG. 3, after insertion, the distal surfaces of the proximal portion 203 of the implant are in close and engaging contact with the walls of recess 201. In an example, insertion of the implant into the intervertebral disk space is halted by contact between the proximal portion 203 of the implant and the walls of recess 201 when the implant has been inserted to the optimal depth within the intervertebral disk space.

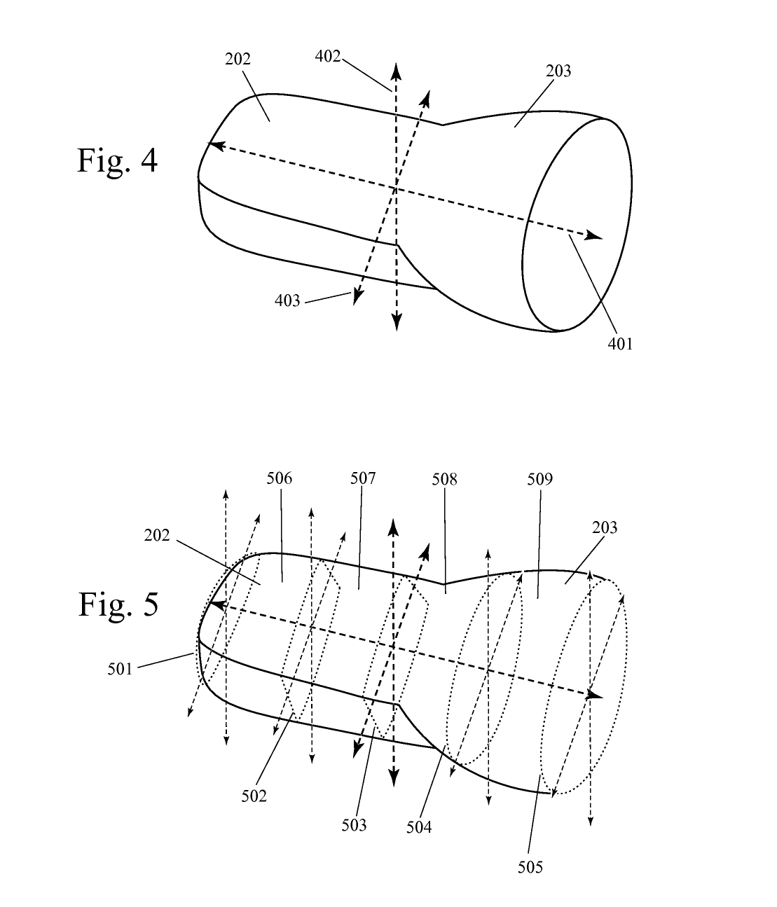

FIGS. 1 through 3 provided a three-stage oblique three-dimensional-solid view of one example of how this invention can be embodied in a device and method for fusing two adjacent spinal vertebrae, including the anatomical context for how the implant is used. FIGS. 4 and 5 now focus more on the geometric specifications of the implant itself. In particular, FIG. 4 shows a cross-sectional view of the implant, including its distal portion 202 and proximal portion 203, with the formal definition of longitudinal, vertical, and horizontal axes for the implant. These axes are then used in FIG. 5 to define segmentation of the implant into four longitudinal segments. These four longitudinal segments are then, in turn, used to precisely specify the unique geometric attributes of the device embodiment of this invention.

The example implant shown in FIGS. 1 through 3 does not have holes for insertion of screws or other fastening members to better attach the implant to the vertebrae. In an example, an implant can have holes for insertion of screws or other fastening members to better attach the implant to the vertebrae. In an example, implants can have holes through which screws are inserted to further attach the implants to the adjacent vertebrae. However, for the purposes of analyzing, categorizing, and specifying basic implant shape and design, we do not include such screws or other fastening member when analyzing and categorizing the basic shape of the implant.

FIG. 4 shows the same implant, with distal portion 202 and proximal portion 203, that was introduced in FIG. 2. FIG. 4 shows the implant without three-dimensional-solid shading (which was pixelated into black-and-white dots to be in conformity with USPTO drawing requirements). This lack of shading shows more clearly the central longitudinal axis 401, central vertical axis 402, and central horizontal axis 403 of the implant.

In the example shown in FIG. 4, a central longitudinal axis 401 (represented by a dotted line with end arrows) is defined for this implant, wherein this central longitudinal axis 401 spans the implant from the distal end (first inserted) to the proximal end (last inserted), wherein this central longitudinal axis 401 is centrally located between the upper surface and the lower surface, wherein this central longitudinal axis 401 is centrally located between the two lateral surfaces, and wherein this central longitudinal axis 401 spans the maximum distance between the distal end and proximal end including any space that is fully or partially enclosed by the walls of the implant. In another and similar example, a central longitudinal axis can be defined as an axis that spans from a distal end (which is first inserted into the intervertebral space) to a proximal end (which is last inserted into the intervertebral space).

In the example shown in FIG. 4, the implant is solid. In an alternative example, FIG. 4 can have holes or windows to promote bone ingrowth and/or complete fusion of the adjacent vertebrae into each other. In another example, FIG. 4 can have a central longitudinal lumen or hole that generally follows central longitudinal axis 401. In an example, a surgical guide wire can be threaded through such a central longitudinal lumen or hole, the guide wire can be inserted into the intervertebral disk space before insertion of the implant, and the implant can then be more easily guided (along the guide wire) into the intervertebral disk space.

In the example shown in FIG. 4, a central vertical axis 402 (represented by a dotted line with end arrows) is defined for this implant, wherein this central vertical axis 402 spans the implant from the lower surface to the top surface, wherein this central vertical axis 402 is perpendicular to the central longitudinal axis, wherein this central vertical axis 402 is centrally located between the distal end and the proximal end, and wherein this central vertical axis 402 is centrally located between the two lateral surfaces. In another and similar example, a central vertical axis can be an axis which is perpendicular to the central longitudinal axis and most parallel to the longitudinal axis of the spine in the section of the two vertebrae.

In the example shown in FIG. 4, a central horizontal axis 403 (represented by a dotted line with end arrows) is defined for this implant, wherein this central horizontal axis 403 spans the implant from one lateral side to the other lateral side, wherein this central horizontal axis 403 is perpendicular to the central longitudinal axis, wherein this central horizontal axis 403 is perpendicular to the central vertical axis, wherein this central horizontal axis 403 is centrally located between the distal end and the proximal end, and wherein this central horizontal axis 403 is centrally located between the lower surface and the upper surface. In another and similar example, a central horizontal axis can be an axis which is perpendicular to the central longitudinal axis and most perpendicular to the longitudinal axis of the spine in the section of the two vertebrae.

FIG. 5 shows an example for the purposes of geometric analysis, not physical construction or separation of the implant, of how the intervertebral implant can be conceptually and longitudinally divided into four segments. This division of the implant into four longitudinal segments can occur as follows. First, the length of the central longitudinal axis 401 is divided into four equal linear portions. Second, three interior lateral cross-sectional areas of the implant are identified, wherein each interior lateral cross-sectional area is parallel to the plane containing the central vertical axis 402 and the central horizontal axis 403 and wherein each interior lateral cross-sectional area contains of the of points along the central longitudinal axis 401 that divides the central longitudinal axis 401 into four equal linear portions. Third, the three interior cross-sectional areas are used to conceptually cut the implant into four longitudinal segments.

The segmentation that is shown in FIG. 5 shows central longitudinal axis 401 having been divided into four equal linear portions. FIG. 5 shows five lateral cross-sectional areas, 501 through 505, that are parallel to the plane containing the central vertical axis 402 and the central horizontal axis 403. Of these five lateral cross-sectional areas, the three interior lateral cross-sectional areas, 502 through 504, conceptually cut the implant into four longitudinal segments, 506 through 509. Segment 506 is the most distal segment. Segment 509 is the most proximal segment.

In this example, lateral cross-sectional areas 502 and 503, located in the distal portion 202 of the implant, are generally rectangular (slightly rounded) in shape. In this example, lateral cross-sectional areas 504 and 505, of the proximal portion 203 of the implant are generally circular in shape. In this example, cross-sectional areas 504 and 505 are generally the same size, reflecting the fact that the distal portion 202 of the implant is generally shaped like a rectangular column (slightly rounded). In this example, cross-sectional area 505 is larger than cross-sectional area 504, reflecting the fact that the proximal portion 203 of the implant is shaped like a section of a cone (e.g. a frustum), not a circular column (e.g. a cylinder).

It is important to note that in this example, division of the implant into four longitudinal segments in FIGS. 4 through 5 is conceptually different than identification of the distal portion 202 and the proximal portion 203 of the implant that was introduced in FIGS. 1 through 3. Conceptual division of the implant into four (generally equal length) longitudinal segments in FIGS. 4 through 5 is based on axially-defined parameters which may, or may not, be correlated with longitudinal differences in the actual structure of the implant. For example, in the embodiment shown here, the proximal portion of the implant comprises approximately one-third of the longitudinal length of the implant, which does not correspond neatly to any single one-fourth length segments or any integer combination of these one-fourth length segments. The independence of the four segments from the physical shape of the implant is intentional. This independence provides an independent and precise framework for specifying the precise geometric parameters and limitations that specify the device embodiment of this invention.

In this example, the four longitudinal segments of the implant are labeled one through four, from the most distal to the most proximal. These numbers are referred to in the narrative but, for diagrammatic consistency, are not the numbers for these segments in the diagram. The first longitudinal segment 506 is the most distal segment of the implant. The second longitudinal segment 507 is the second-most distal segment of the implant. The third longitudinal segment 508 is the second-most proximal segment of the implant. The fourth longitudinal segment 509 is the most proximal segment of the implant.

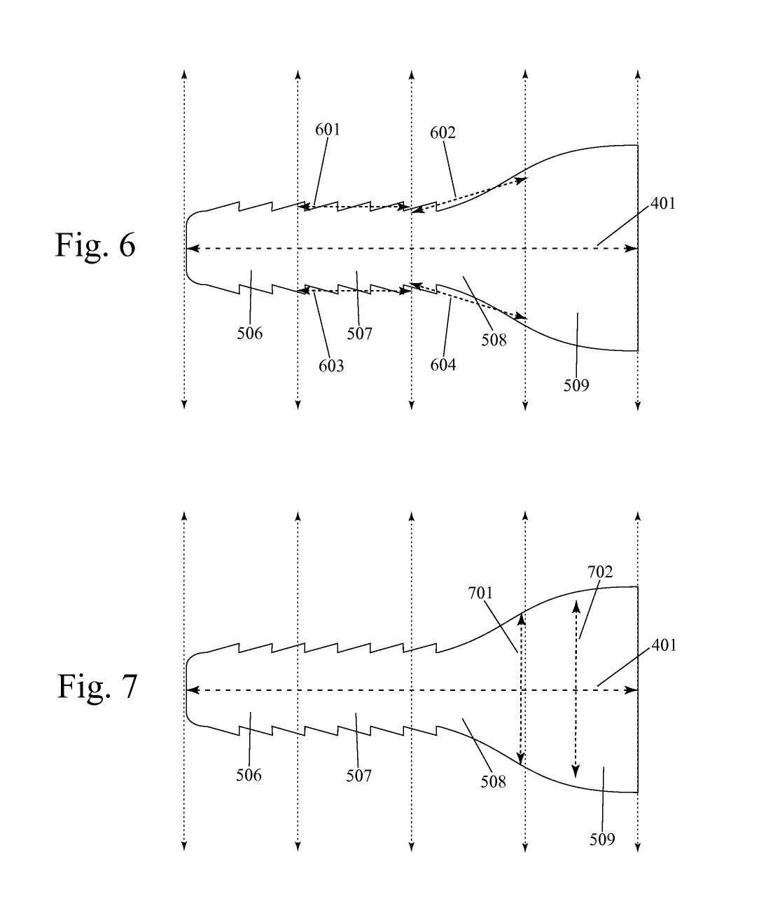

FIGS. 6 and 7 show vertical cross-sectional views of an example of how this invention can be embodied in an intervertebral implant comprising: a distal portion that is shaped like a rectangular column with rounded edges and with ridges on the upper and lower surfaces; and a proximal portion that is shaped like a section of a convex cone. In this example, the distal portion of the implant comprises approximately 60%-70% of the longitudinal length of the implant and the distal portion comprises the remaining portion of this length.

FIGS. 6 and 7 also show how the axial framework for the implant and the longitudinal segmentation of the implant that were introduced in FIGS. 4 and 5 can be used to precisely specify the geometric features of the device embodiment of this invention. FIGS. 6 and 7 show a central longitudinal axis 401 of this implant. FIGS. 6 and 7 also show how implant has been divided into four longitudinal segments, 506 through 509.

In an example, a maximum-height longitudinal cross-sectional area can be defined for each of the four segments, 506 through 509, wherein each longitudinal cross-sectional area is parallel to the plane containing the central longitudinal axis and the central vertical axis, and wherein the maximum-height longitudinal cross-sectional area for a segment is that longitudinal cross-sectional area which contains the maximum distance between the lower surface and upper surface as measured along a vector that is parallel to the central vertical axis. For the first and fourth segments, 506 and 509, the longitudinal cross-sectional area can be defined as between a cross-sectional and an end of the implant.

In an example, an upper perimeter can also be defined for each of the four segments, 506 through 509, wherein the upper perimeter is the upper portion of the maximum-height longitudinal cross-sectional area that is between the lateral cross-sectional areas that separate segments. In an example, a lower perimeter can be defined for each of the four segments, wherein the lower perimeter is the lower portion of the maximum-height longitudinal cross-sectional area that is between the lateral cross-sectional areas that separate segments.

In an example, a segment maximum height can be defined for each segment, 506 through 509, wherein the maximum height is the maximum distance between the segment's upper perimeter and lower perimeter as measured along a vector that is parallel to the central vertical axis. In an example, a segment average height can be defined for each segment, wherein the average height is the average distance between the segment's upper perimeter and lower perimeter as measured along vectors that are parallel to the central vertical axis.

In an example, a best-fitting straight line can be defined for the upper perimeter of a segment and a best-fitting straight line can be defined for the lower perimeter of a segment. In an example, a best-fitting straight line for a perimeter can be the straight line that minimizes the sum of squared deviations from the points along this perimeter. In an example, a best-fitting straight line for a perimeter can be the straight line that minimizes the sum of the absolute values of deviations from the points along this perimeter. In an example, a best-fitting straight line for a perimeter can be the straight line that best fits the perimeter after one removes or cancels repeated wave patterns or oscillations along the perimeter that are associated with a repeated pattern of ridges, protrusions, or holes.

In the example shown in FIG. 6, line 601 is the best-fitting straight line for the upper perimeter of second longitudinal segment 507 and line 602 is the best-fitting straight line for the upper perimeter of third longitudinal segment 508. In the example shown in FIG. 6, line 603 is the best-fitting straight line for the lower perimeter of second longitudinal segment 507 and line 604 is the best-fitting straight line for the lower perimeter of third longitudinal segment 508.

In the example shown in FIG. 6, best-fitting line 601 for the upper perimeter of the second longitudinal segment is substantially parallel to best-fitting line 603 for the lower perimeter of the second longitudinal segment. Also, in the example shown in FIG. 6, best-fitting line 602 for the upper perimeter of the third longitudinal segment diverges from best-fitting line 604 for the lower perimeter of the third longitudinal segment with distal-to-proximal movement along the perimeters.

In an example, a segment upper slope can be defined as the slope of the best-fitting straight line for the segment's upper perimeter, wherein slope is defined as vertical change divided by longitudinal change when moving in a distal-to-proximal direction. In an example, a segment lower slope can be defined as the slope of the best-fitting straight line for the segment's lower perimeter, wherein slope is defined as vertical change divided by longitudinal change when moving in a distal-to-proximal direction.

In the example shown in FIG. 6, the segment upper slope of the second longitudinal segment 507 (which is the slope of line 601) is zero and the segment upper slope of the third longitudinal segment 508 (which is the slope of line 602) is positive. In the example shown in FIG. 6, the segment lower slope of the second longitudinal segment 507 (which is the slope of line 603) is zero and the segment lower slope of the third longitudinal segment 508 (which is the slope of line 604) is negative.

Also, in the example shown in FIG. 6, the segment upper slope of the third longitudinal segment 508 (which is the slope of line 602) is more positive than the segment upper slope of the second longitudinal segment 507 (which is the slope of line 601). Also, in the example shown in FIG. 6, the segment lower slope of the third longitudinal segment 508 (which is the slope of line 604) is more negative than the segment lower slope of the second longitudinal segment 507 (which is the slope of line 603).

In an example, this invention can be embodied in an implant wherein one or more of the conditions selected from the following group apply: the segment upper slope of longitudinal segment three 508 is more positive than the segment upper slope of segment two 507; and the segment lower slope of segment three 508 is more negative than the segment lower slope of segment two 507.

As shown in FIG. 7, this implant can be further specified such that the segment average height of longitudinal segment four 509 is no less than the segment maximum height of longitudinal segment three 508. FIG. 7 shows the segment maximum height 701 of longitudinal segment three 508. In FIG. 7, this maximum height is represented by dotted line with end arrows 701. FIG. 7 also shows the segment average height 702 of longitudinal segment four 509. In FIG. 7, this average height is represented by dotted line with end arrows 702. As shown in FIG. 7, the average height 702 of segment four is no less than the maximum height 701 of segment three.

In an more restrictive example, this invention can be embodied in an implant for which one or more of the conditions selected from the following group can apply: the segment upper slope of segment three is at least 25% more positive than the segment upper slope of segment two; and the segment lower slope of segment three is at least 25% more negative than the segment lower slope of segment two. In another restrictive example, this invention can be embodied in an implant for which one or more of the conditions selected from the following group can apply: the segment upper slope of segment four is at least 25% more positive than the segment upper slope of segment two; and the segment lower slope of segment four is at least 25% more negative than the segment lower slope of segment two.

In an example, a central longitudinal axis of an intervertebral implant can be divided into four equal lengths and the three cross-sectional areas that separate these four equal lengths also separate four longitudinal segments. In an example, the average height of the cross-sections that comprise a third segment of an implant can be greater than the maximum height of the cross-sections that comprise the second segment of an implant. In an example, the average height of the cross-sections that comprise the fourth segment of an implant can be greater than the maximum height of the cross-sections that comprise the third segment of an implant.

In an alternative example, an upper linear perimeter of a segment can be defined as the straight line that best fits the uppermost points of the cross-sections in that segment, wherein the best fitting line is the line that minimizes the sum of squared deviations from points along the perimeter. Similarly, a lower linear perimeter of a segment can be defined as the straight line that best fits the lowermost points of the cross-sections in that segment, wherein the best fitting line is the line that minimizes the sum of squared deviations from points along the perimeter.

In an example, an upper linear perimeter of a segment can be defined as the straight line that best fits the uppermost points of the cross-sections in that segment, wherein the best fitting line is the line that minimizes the sum of absolute values of deviations from points along the perimeter. In an example, the lower linear perimeter of a segment can be defined as the straight line that best fits the lowermost points of the cross-sections in that segment, wherein the best fitting line is the line that minimizes the sum of absolute values of deviations from points along the perimeter.

In an example, the slope of the upper linear perimeter of the third segment can be more positive than the slope of the upper linear perimeter of the second segment, moving in a distal-to-proximal direction, wherein slope is vertical change divided by longitudinal change. In an example, the slope of the lower linear perimeter of the third segment can be more negative than the slope of the lower linear perimeter of the second segment, moving in a distal-to-proximal direction, wherein slope is vertical change divided by longitudinal change.

In an example, the slope of the upper linear perimeter of the fourth segment can be more positive than the slope of the upper linear perimeter of the third segment, moving in a distal-to-proximal direction, wherein slope is vertical change divided by longitudinal change. In an example, the slope of the lower linear perimeter of the fourth segment can be more negative than the slope of the lower linear perimeter of the third segment, moving in a distal-to-proximal direction, wherein slope is vertical change divided by longitudinal change.

In an example, the slope of the upper linear perimeter of the second segment and the slope of the lower linear perimeter of the second segment can both be substantially zero, but the slope of the upper linear perimeter of the third segment can be positive and the slope of the lower linear perimeter of the third segment can be negative.

In an example, the distance between the upper linear perimeter of the second segment and the lower linear perimeter of the second segment can remain constant as one moves in a distal-to-proximal direction, but the distance between the upper linear perimeter of the third segment and the lower linear perimeter of the third segment can increase as one moves in a distal-to-proximal direction.

In an example, the distance between the upper linear perimeter of a second segment and the lower linear perimeter of a second segment can remain constant as one moves in a distal-to-proximal direction, but the distance between the upper linear perimeter of a third segment and the lower linear perimeter of the third segment can increase as one moves in a distal-to-proximal direction. Further, the distance between the upper linear perimeter of the fourth segment and the lower linear perimeter of the fourth segment can increase as one moves in a distal-to-proximal direction.

In an example, the distance between the upper linear perimeter of the second segment and the lower linear perimeter of the second segment can remain constant as one moves in a distal-to-proximal direction, but the distance between the upper linear perimeter of the third segment and the lower linear perimeter of the third segment can increase in a non-linear manner as one moves in a distal-to-proximal direction. Further, the distance between the upper linear perimeter of the fourth segment and the lower linear perimeter of the fourth segment can increase in a non-linear manner as one moves in a distal-to-proximal direction.

In an example, the distance between the upper linear perimeter of the second segment and the lower linear perimeter of the second segment can remain constant as one moves in a distal-to-proximal direction, but the distance between the upper linear perimeter of the third segment and the lower linear perimeter of the third segment can increase in a greater-than-linear manner as one moves in a distal-to-proximal direction. Further, the distance between the upper linear perimeter of the fourth segment and the lower linear perimeter of the fourth segment can increase in a greater-than-linear manner as one moves in a distal-to-proximal direction.

In an example, the distance between the upper linear perimeter of the second segment and the lower linear perimeter of the second segment can remain constant as one moves in a distal-to-proximal direction, but the distance between the upper linear perimeter of the third segment and the lower linear perimeter of the third segment can increase in a less-than-linear manner as one moves in a distal-to-proximal direction. Further, the distance between the upper linear perimeter of the fourth segment and the lower linear perimeter of the fourth segment can increase in a less-than-linear manner as one moves in a distal-to-proximal direction.

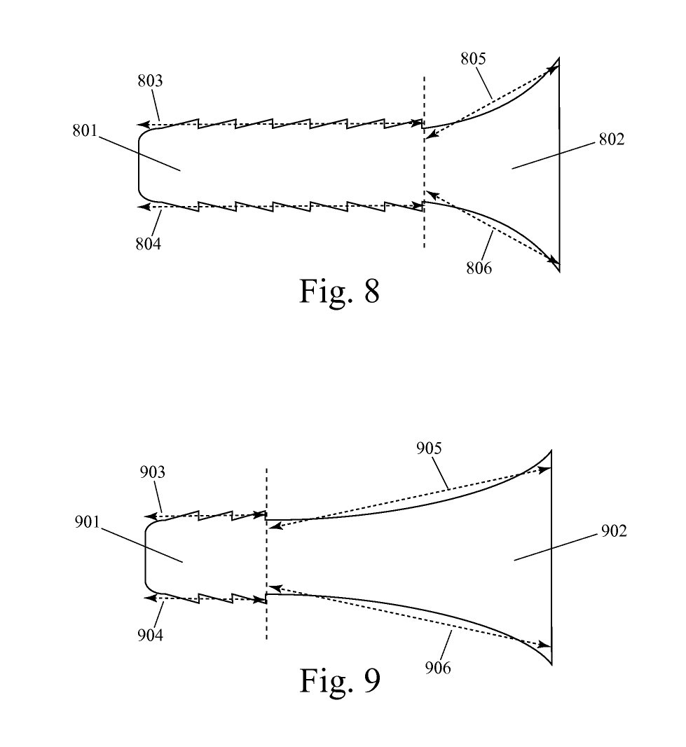

FIG. 8 shows an example of how this invention can be embodied in an intervertebral implant comprising: a distal portion 801 that is shaped like a rectangular column with rounded edges and ridged upper and lower surfaces; and a proximal portion 802 that is shaped like a section (i.e. frustum) of a cone with concave sides from the cone base to the cone peak. In this example, the distal portion 801 comprises approximately two-thirds of the longitudinal length of the implant and the proximal 802 comprises approximately one-third of the longitudinal length of the implant.

In an example, a best-fitting flat plane for a surface can be defined as the flat plane that minimizes the sum of squared deviations from the points on that surface. In an example, a best-fitting flat plane for a surface can be defined as the flat plane that minimizes the sum of absolute values of deviations from the points on that surface. In an example, a best-fitting flat plane for a surface can be defined as the flat plane that remains if one were to geometrically subtract or cancel a repeating sequence of ridges, protrusions, or holes from that surface.

In the example shown in FIG. 8, a best-fitting flat plane 803 can be defined for the upper surface of the distal portion 801 of the implant. Also, a best-fitting flat plane 804 can be defined for the lower surface of the distal portion 801 of the implant. In the example shown in FIG. 8, the best-fitting flat plane 803 for the upper surface of the distal portion 801 of the implant is substantially parallel to the best-fitting flat plane 804 for the lower surface of the distal portion 801 of the implant. In this example, the distal portion 801 of the implant is shaped substantially like a rectangular column, albeit with slightly rounded edges and a repeated pattern of ridges on the upper and lower surfaces.

In the example shown in FIG. 8, a best-fitting flat plane 805 can also be defined for the upper surface of the proximal portion 802 of the implant. Also, a best-fitting flat plane 806 can be defined for the lower surface of the proximal portion 802 of the implant. In the example shown in FIG. 8, the best-fitting flat plane 805 for the upper surface of the proximal portion 802 of the implant diverges from the best-fitting flat plane 806 for the lower surface of the distal portion 802 of the implant as one moves in a distal-to-proximal direction. In this example, the proximal portion 802 of the implant is shaped substantially like a section of a cone with concave sides.

The example shown in FIG. 9 is similar to the example shown in FIG. 8, except that the distal portion 901 comprises approximately one-third of the longitudinal length of the implant and the proximal portion 902 comprises approximately two-thirds of the longitudinal length of the implant.

As was done with FIG. 8, in the example shown in FIG. 9 a best-fitting flat plane 903 can be defined for the upper surface of the distal portion 901 of the implant. Also, a best-fitting flat plane 904 can be defined for the lower surface of the distal portion 901 of the implant. The best-fitting flat plane 903 for the upper surface of the distal portion 901 of the implant is substantially parallel to the best-fitting flat plane 904 for the lower surface of the distal portion 901 of the implant. In this example, the distal portion 901 of the implant is shaped substantially like a rectangular column, albeit with slightly rounded edges and a repeated pattern of ridges on the upper and lower surfaces.

In the example shown in FIG. 9, a best-fitting flat plane 905 can also be defined for the upper surface of the proximal portion 902 of the implant. Also, a best-fitting flat plane 906 can be defined for the lower surface of the proximal portion 902 of the implant. The best-fitting flat plane 905 for the upper surface of the proximal portion 902 of the implant diverges from the best-fitting flat plane 906 for the lower surface of the distal portion 902 of the implant as one moves in a distal-to-proximal direction. In this example, the proximal portion 902 of the implant is shaped substantially like a section of a cone with concave sides.

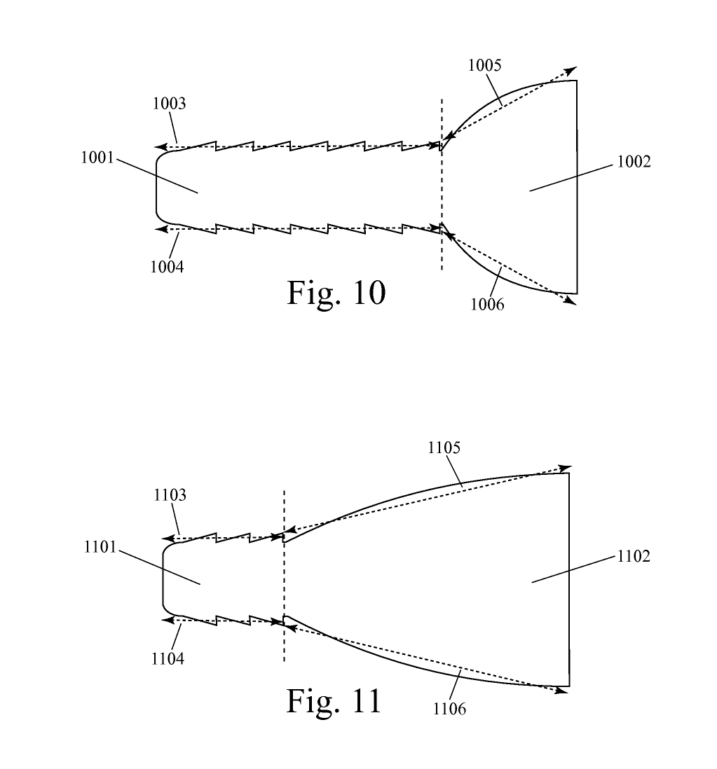

FIGS. 10 and 11 show examples of this invention that are similar to the examples shown in FIGS. 8 and 9 except that now the proximal portion of the implant is a section of a cone with convex, rather than concave, walls. As we discussed earlier, the relative concavity or convexity of the surface of the proximal portion can be optimized to facilitate insertion of the implant from an anatomically-restricted range of entry angles. For example, insertion of the implant into lower vertebrae may be restricted by the presence of nerves or muscles to less-direct and larger entry angles and such insertion may be facilitated by greater convexity or concavity of the proximal portion and/or the corresponding convexity or concavity of the recess drilled into the vertebrae.

The example of an intervertebral implant that is shown in FIG. 10 comprises: a distal portion 1001 that is shaped like a rectangular column with rounded edges and ridged upper and lower surfaces; and a proximal portion 1002 that is shaped like a section (i.e. frustum) of a cone with convex sides from the cone base to the cone peak. In this example, the distal portion 1001 comprises approximately two-thirds of the longitudinal length of the implant and the proximal 1002 comprises approximately one-third of the longitudinal length of the implant.

In FIG. 10, a best-fitting flat plane 1003 can be defined for the upper surface of the distal portion 1001 of the implant. Also, a best-fitting flat plane 1004 can be defined for the lower surface of the distal portion 1001 of the implant. In FIG. 10, the best-fitting flat plane 1003 for the upper surface of the distal portion 1001 of the implant is substantially parallel to the best-fitting flat plane 1004 for the lower surface of the distal portion 1001 of the implant. In this example, the distal portion 1001 of the implant is shaped substantially like a rectangular column, albeit with slightly rounded edges and a repeated pattern of ridges on the upper and lower surfaces.

In FIG. 10, a best-fitting flat plane 1005 can also be defined for the upper surface of the proximal portion 1002 of the implant. Also, a best-fitting flat plane 1006 can be defined for the lower surface of the proximal portion 1002 of the implant. In FIG. 10, the best-fitting flat plane 1005 for the upper surface of the proximal portion 1002 of the implant diverges from the best-fitting flat plane 1006 for the lower surface of the distal portion 1002 of the implant as one moves in a distal-to-proximal direction. In this example, the proximal portion 1002 of the implant is shaped substantially like a section of a cone with convex sides.

The example shown in FIG. 11 is similar to the example shown in FIG. 10, except that the distal portion 1101 comprises approximately one-third of the longitudinal length of the implant and the proximal portion 1102 comprises approximately two-thirds of the longitudinal length of the implant. In FIG. 11 a best-fitting flat plane 1103 can be defined for the upper surface of the distal portion 1101 of the implant. Also, a best-fitting flat plane 1104 can be defined for the lower surface of the distal portion 1101 of the implant. The best-fitting flat plane 1103 for the upper surface of the distal portion 1101 of the implant is substantially parallel to the best-fitting flat plane 1104 for the lower surface of the distal portion 1101 of the implant. In this example, the distal portion 1101 of the implant is shaped substantially like a rectangular column, albeit with slightly rounded edges and a repeated pattern of ridges on the upper and lower surfaces.

In the example shown in FIG. 11, a best-fitting flat plane 1105 can also be defined for the upper surface of the proximal portion 1102 of the implant. Also, a best-fitting flat plane 1106 can be defined for the lower surface of the proximal portion 1102 of the implant. The best-fitting flat plane 1105 for the upper surface of the proximal portion 1102 of the implant diverges from the best-fitting flat plane 1106 for the lower surface of the distal portion 1102 of the implant as one moves in a distal-to-proximal direction. In this example, the proximal portion 1102 of the implant is shaped substantially like a section of a cone with convex sides.

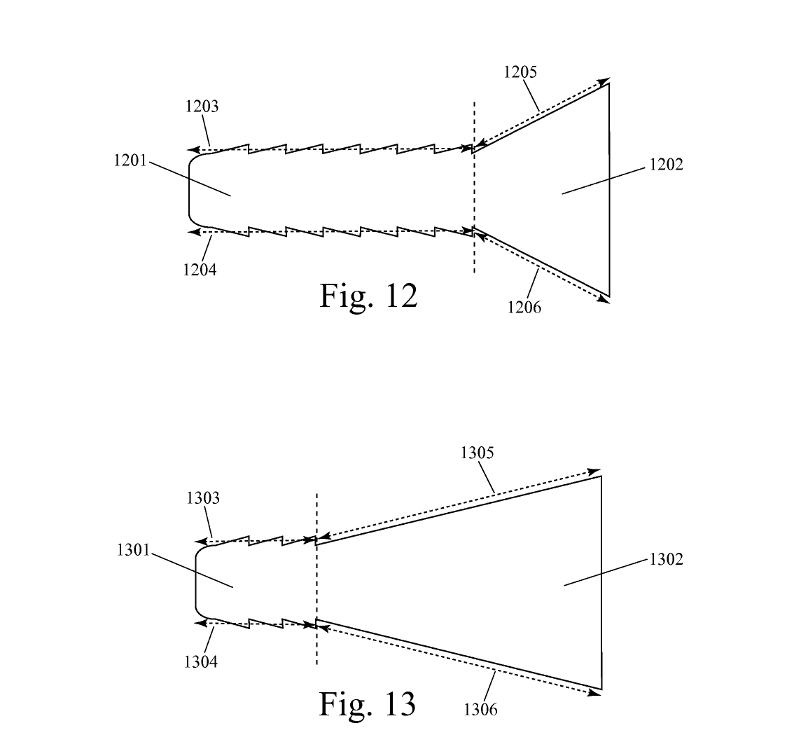

FIGS. 12 and 13 show examples of this invention that are similar to the examples shown in FIGS. 8 and 9, except that now the proximal portion of the implant is a section of a cone with straight walls. The example of an intervertebral implant that is shown in FIG. 12 comprises: a distal portion 1201 that is shaped like a rectangular column with rounded edges and ridged upper and lower surfaces; and a proximal portion 1202 that is shaped like a section (i.e. frustum) of a cone with straight sides from the cone base to the cone peak. In this example, the distal portion 1201 comprises approximately two-thirds of the longitudinal length of the implant and the proximal 1202 comprises approximately one-third of the longitudinal length of the implant.

In FIG. 12, a best-fitting flat plane 1203 can be defined for the upper surface of the distal portion 1201 of the implant. Also, a best-fitting flat plane 1204 can be defined for the lower surface of the distal portion 1201 of the implant. In FIG. 12, the best-fitting flat plane 1203 for the upper surface of the distal portion 1201 of the implant is substantially parallel to the best-fitting flat plane 1204 for the lower surface of the distal portion 1201 of the implant. In this example, the distal portion 1201 of the implant is shaped substantially like a rectangular column, albeit with slightly rounded edges and a repeated pattern of ridges on the upper and lower surfaces.

In FIG. 12, a best-fitting flat plane 1205 can also be defined for the upper surface of the proximal portion 1202 of the implant. Also, a best-fitting flat plane 1206 can be defined for the lower surface of the proximal portion 1202 of the implant. In FIG. 12, the best-fitting flat plane 1205 for the upper surface of the proximal portion 1202 of the implant diverges from the best-fitting flat plane 1206 for the lower surface of the distal portion 1202 of the implant as one moves in a distal-to-proximal direction. In this example, the proximal portion 1202 of the implant is shaped substantially like a section of a cone with straight sides.

The example shown in FIG. 13 is similar to example shown in FIG. 12, except that the distal portion 1301 comprises approximately one-third of the longitudinal length of the implant and the proximal portion 1302 comprises approximately two-thirds of the longitudinal length of the implant. In FIG. 13 a best-fitting flat plane 1303 can be defined for the upper surface of the distal portion 1301 of the implant. Also, a best-fitting flat plane 1304 can be defined for the lower surface of the distal portion 1301 of the implant. The best-fitting flat plane 1303 for the upper surface of the distal portion 1301 of the implant is substantially parallel to the best-fitting flat plane 1304 for the lower surface of the distal portion 1301 of the implant. In this example, the distal portion 1301 of the implant is shaped substantially like a rectangular column, albeit with slightly rounded edges and a repeated pattern of ridges on the upper and lower surfaces.

In the example shown in FIG. 13, a best-fitting flat plane 1305 can also be defined for the upper surface of the proximal portion 1302 of the implant. Also, a best-fitting flat plane 1306 can be defined for the lower surface of the proximal portion 1302 of the implant. The best-fitting flat plane 1305 for the upper surface of the proximal portion 1302 of the implant diverges from the best-fitting flat plane 1306 for the lower surface of the distal portion 1302 of the implant as one moves in a distal-to-proximal direction. In this example, the proximal portion 1302 of the implant is shaped substantially like a section of a cone with straight sides.

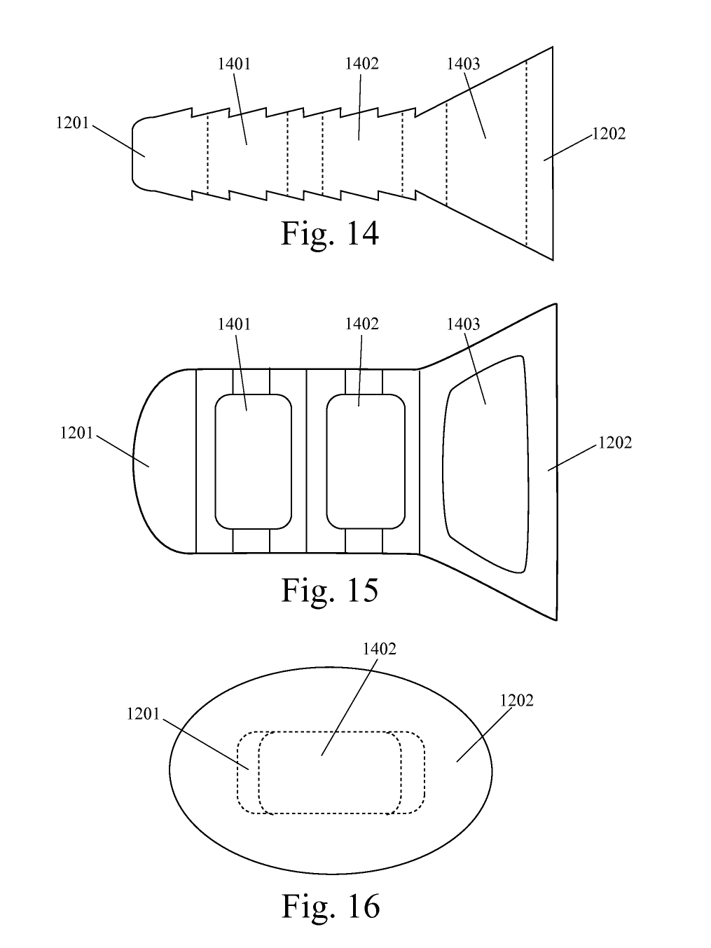

FIGS. 14 through 16 show three views, from three different perspectives, of an example of how this invention can be embodied in an intervertebral implant comprising: a distal portion 1201 that is shaped like a rectangular column with rounded edges, ridges on its upper and lower surfaces, and two holes, 1401 and 1402, between its upper and lower surfaces; and a proximal portion 1202 that is shaped like a section of a cone with an elliptical base and straight walls from its base to its peak and which has a hole, 1403, between its upper and lower surfaces. In this example, the distal portion 1201 of the implant spans approximately two-thirds of the longitudinal length of the implant and the proximal portion 1202 of the implant spans the remaining one-third of this length.

FIG. 14 shows a lateral side view of this example of an intervertebral implant. From this perspective, one can see the shape of the longitudinal cross-section of this implant, including the longitudinal cross-sectional shapes of the distal portion 1201 and the proximal portion 1202. The ridges along the upper and lower surfaces of the distal portion 1201 are clearly seen. The walls of the three holes, 1401 through 1403, which span between the upper and lower surfaces of the distal and proximal portions, are shown by dotted lines from this perspective because they are hidden from view beneath the lateral sides of the implant from this perspective.

FIG. 15 shows a top view of this example of an intervertebral implant. From this perspective, one can see the shape of the lateral cross-section of this implant, including the lateral cross-sectional shapes of the distal portion 1201 and the proximal portion 1202. The ridges of distal portion 1201 are only seen as lines that laterally span the upper surface of the distal portion 1201. The three holes, 1401 through 1403, are now directly visible and clearly shown.

FIG. 16 shows a proximal end view of this example of an intervertebral implant. From this perspective, one can only directly see the elliptical shape of the proximal end 1202 of the implant. The generally rectangular cross sectional shape of the distal portion is not directly seen, but rather shown as a dotted line rounded rectangle 1201. Hole 1402 is also represented by a dotted line perimeter.

FIGS. 17 through 19 show another example of how this invention can be embodied. This example, and the three views thereof, are similar to that shown in FIGS. 14 through 16 except that in this example the implant has a trapezoidal vertical horizontal cross-sectional shape which is designed for implantation between two vertebrae with lordosis. Lordosis is anterior concavity in the curvature of the spine as viewed from the side. In this respect, this example of the invention can be said to have a “lordotic shape” and can be useful for side implantation between vertebrae in a lordotic section of the spine.

FIGS. 17 through 19 show an intervertebral implant comprising: a lordotic distal portion 1701 that is shaped like a trapezoidal column with rounded edges, ridges on its upper and lower surfaces, and two holes, 1703 and 1704, between its upper and lower surfaces; and a proximal portion 1702 that is shaped like a section of a cone with an elliptical base and straight walls from its base to its peak and which has a hole, 1705, between its upper and lower surfaces. In this example, the distal portion 1701 of the implant spans approximately two-thirds of the longitudinal length of the implant and the proximal portion 1702 of the implant spans the remaining one-third of this length.

FIG. 17 shows a lateral side view of this example of a lordotic intervertebral implant. From this perspective, one can see the shape of the longitudinal cross-section of this implant, including the longitudinal cross-sectional shapes of the distal portion 1701 and the proximal portion 1702. The ridges along the upper and lower surfaces of the distal portion 1701 are clearly seen. The walls of the three holes, 1703 through 1705, which span between the upper and lower surfaces of the distal and proximal portions, are shown by dotted lines from this perspective because they are hidden from view beneath the lateral sides of the implant from this perspective.

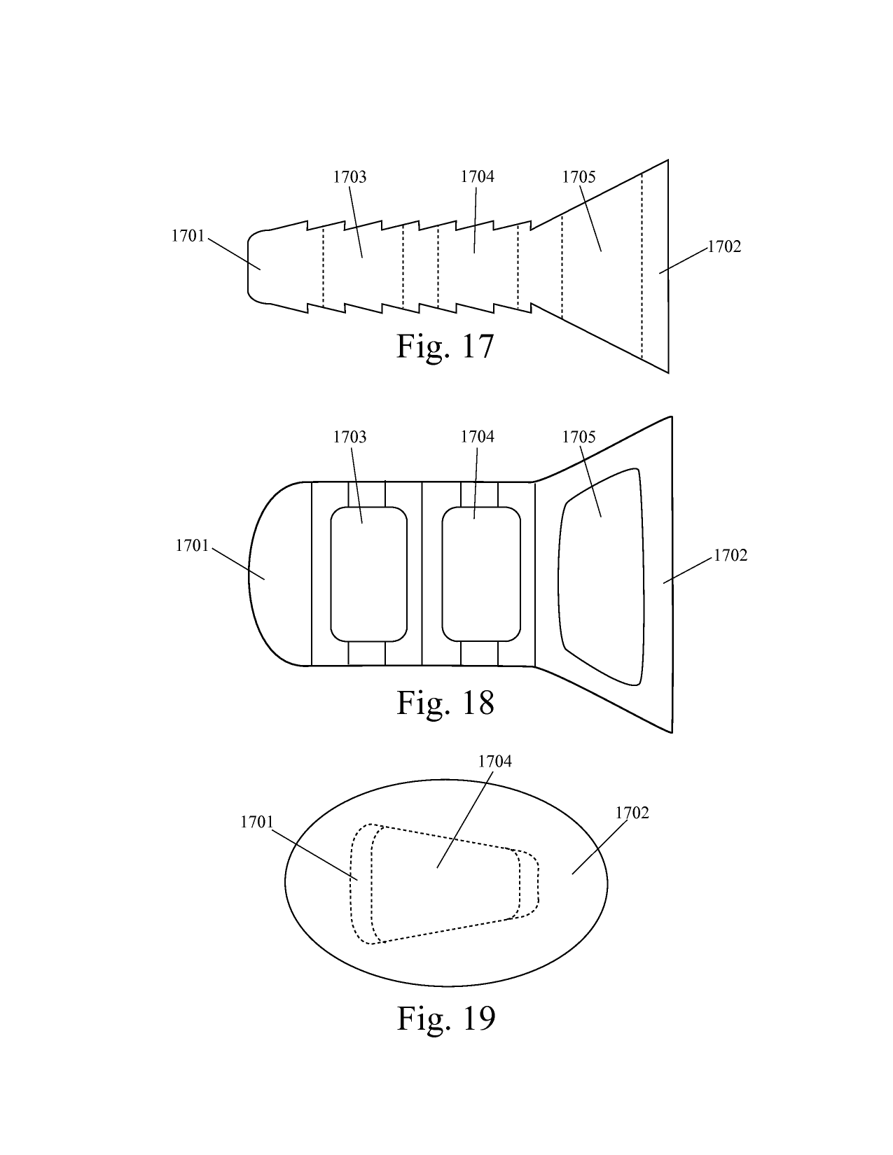

FIG. 18 shows a top view of this example of a lordotic intervertebral implant. From this perspective, one can see the shape of the lateral cross-section of this implant, including the lateral cross-sectional shapes of the distal portion 1701 and the proximal portion 1702. The ridges of distal portion 1701 are only seen as lines that laterally span the upper surface of the distal portion 1701. The three holes, 1703 through 1705, are now directly visible and clearly shown.

FIG. 19 shows a proximal end view of this example of a lordotic intervertebral implant. From this perspective, one can only directly see the elliptical shape of the proximal end 1702 of the implant. The generally trapezoidal cross sectional shape of the distal portion is not directly seen, but rather shown as a dotted line rounded trapezoid 1701. Hole 1704 is also represented by a dotted line perimeter.

FIGS. 1 through 19 also show how this invention can be embodied in a method for fusing spinal vertebrae. In an example, this method can comprise: (a) drilling a recess into a section of the spine comprising two adjacent spinal vertebrae; wherein this recess includes a portion of the intervertebral disk space, a portion of the upper vertebrae that is contiguous the intervertebral disk space, and a portion of the lower vertebrae that is contiguous the intervertebral disk space; wherein this recess extends between 25% and 75% of the lateral span of the intervertebral disk space; and wherein this recess is shaped like a section of a cone or rotated polygon; and wherein this recess has a wider proximal cross-section than distal cross-section; and (b) inserting an intervertebral implant into the intervertebral disk space and recess such that the distal end of the implant is substantially flush with the surface of the vertebrae on the side of the spinal column opposite the recess and the proximal end of the implant is substantially flush with the pre-drilling surface of the spinal column on the side of the vertebrae that has the recess.

In an example, the proximal surface of the intervertebral implant can substantially conform to the wall of the recess when the intervertebral implant is inserted into the intervertebral space. In an example, the curved walls of the recess can help to guide the distal end of the intervertebral implant into the intervertebral space from a variety of insertion angles. This can be an improvement over the prior art in which an implant is difficult to insert from other than straight-line entry. In an example, the curved walls of the recess can guide the distal end of the implant into the intervertebral space from a variety of entry angles.

In an example, the distal surface of the proximal portion of the implant can substantially conform to the walls of the recess as a whole. In an example, the implant can fit substantially flush with the lateral surfaces of the vertebrae when the distal surface of the proximal portion fits flush into the contour of the recess. In an example, in addition to guiding the distal end of the implant into the intervertebral space, the recess walls can also guide the proper insertion depth of the implant. In an example, insertion of the implant stops at the desired insertion depth when the distal surface of the proximal portion of the implant comes into conformal contact with the walls of the recess.

FIGS. 1 through 19 show examples of how this invention can be embodied in an intervertebral implant for fusing spinal vertebrae comprising: an implant that is implanted into the intervertebral disk space between two spinal vertebrae, wherein the following specifications apply to the implant excluding any fastening members which can be rotated or slid inwards independently of the implant; the implant further comprising a distal portion that is first inserted into the intervertebral disk space, wherein this distal portion has a rounded distal end, two lateral surfaces, an upper surface, and a lower surface, wherein the best-fitting flat plane for the upper surface and the best-fitting flat plane for the lower surface are substantially parallel to each other, wherein the best-fitting flat plane for a surface is the flat plane that minimizes the sum or squared deviations from points on the surface; and wherein this distal portion spans at least 25% and no more than 75% of the distal-to-proximal length of the implant; and the implant further comprising a proximal portion, wherein this proximal portion has an upper surface and a lower surface, wherein the best-fitting flat plane for the upper surface and the best-fitting flat plane for the lower surface are further apart at the proximal end of the proximal portion than they are at the distal end of the proximal portion, wherein the best-fitting flat plane for a surface is the flat plane that minimizes the sum or squared deviations from points on the surface, and wherein this proximal portion spans the remaining length of the distal-to-proximal length after accounting for the distal portion.

FIGS. 1 through 19 also show examples of how this invention can be embodied in a device wherein the distal portion spans between 25% and 50% of the distal-to-proximal length of the implant and the proximal portion spans the remaining portion of the distal-to-proximal length of the implant. FIGS. 1 through 19 also show examples of how this invention can be embodied in a device wherein the distal portion spans between 50% and 75% of the distal-to-proximal length of the implant and the proximal portion spans the remaining portion of the distal-to-proximal length of the implant.

FIGS. 1 through 19 also show examples of how this invention can be embodied in a device wherein the distal portion is shaped substantially like a rectangular column with substantially parallel upper and lower surfaces, with the possible exception of having rounded edges and a plurality of ridges or other protrusions on its upper and lower surfaces. FIGS. 1 through 19 also show examples of how this invention can be embodied in a device wherein the distal portion is shaped substantially like a trapezoidal column with substantially parallel side surfaces. FIGS. 1 through 19 also show examples of how this invention can be embodied in a device wherein the distal portion is shaped substantially like an elliptical column with a plurality of ridges or other protrusions on its upper and lower surfaces.

FIGS. 1 through 19 also show examples of how this invention can be embodied in a device wherein the proximal portion is shaped substantially like a section of a cone that has a circular base and straight sides from the cone base to the peak. FIGS. 1 through 19 also show examples of how this invention can be embodied in a device wherein the proximal portion is shaped substantially like a section of a cone that has a circular base and convex sides from the cone base to the peak. FIGS. 1 through 19 also show examples of how this invention can be embodied in a device wherein the proximal portion is shaped substantially like a section of a cone that has a circular base and concave sides from the cone base to the peak.

FIGS. 1 through 19 also show examples of how this invention can be embodied in a device wherein the proximal portion is shaped substantially like a section of a cone that has a elliptical base and straight sides from the cone base to the peak. FIGS. 1 through 19 also show examples of how this invention can be embodied in a device wherein the proximal portion is shaped substantially like a section of a cone that has a elliptical base and convex sides from the cone base to the peak. FIGS. 1 through 19 also show examples of how this invention can be embodied in a device wherein the proximal portion is shaped substantially like a section of a cone that has a elliptical base and concave sides from the cone base to the peak.

In another example, this invention can be embodied in a device wherein the proximal portion is shaped substantially like a section of a rotated polygon. In another example, this invention can be embodied in a device wherein the proximal portion is shaped substantially like a section of a sphere.

FIGS. 1 through 19 also show examples of how this invention can be embodied in a device wherein there are a plurality of ridges or other protrusions on the upper surface of the implant and/or on the lower surface of the implant in order to promote bone ingrowth and/or attachment of the implant to the vertebrae. FIGS. 1 through 19 also show examples of how this invention can be embodied in a device wherein there are a plurality of holes in the upper surface of the implant, in the lower surface of the implant, or extending from the upper surface of the implant to the lower surface of the implant in order to promote bone ingrowth, attachment of the implant to the vertebrae, and/or complete fusion of the vertebrae to each other.

FIGS. 1 through 19 also show examples of how this invention can be embodied in an intervertebral implant for fusing spinal vertebrae comprising: an implant that is implanted into the intervertebral disk space between two spinal vertebrae, wherein the following specifications apply to the implant excluding any fastening members which can be rotated and/or inserted inwards independently of the implant; wherein the implant comprises a distal end, a proximal end, an upper surface, a lower surface, and two lateral surfaces, and wherein the distal end is the end that is first implanted into the intervertebral disk space; wherein a central longitudinal axis can be defined for this implant, wherein this central longitudinal axis spans the implant from the distal end to the proximal end, wherein this central longitudinal axis is centrally located between the upper surface and the lower surface, wherein this central longitudinal axis is centrally located between the two lateral surfaces, and wherein this central longitudinal axis spans the maximum distance between the distal end and proximal end including any space that is fully or partially enclosed by the walls of the implant; wherein a central vertical axis can be defined for this implant, wherein this central vertical axis spans the implant from the lower surface to the top surface, wherein this central vertical axis is perpendicular to the central longitudinal axis, wherein this central vertical axis is centrally located between the distal end and the proximal end, and wherein this central vertical axis is centrally located between the two lateral surfaces; wherein a central horizontal axis can be defined for this implant, wherein this central horizontal axis spans the implant from one lateral side to the other lateral side, wherein this central horizontal axis is perpendicular to the central longitudinal axis, wherein this central horizontal axis is perpendicular to the central vertical axis, wherein this central horizontal axis is centrally located between the distal end and the proximal end, and wherein this central horizontal axis is centrally located between the lower surface and the upper surface; wherein the implant can be longitudinally divided into four segments, wherein the length of the central longitudinal axis is divided into four equal linear portions, wherein there are three lateral cross-sectional areas separating these four equal linear portions, wherein each lateral cross-sectional area is parallel to the plane containing the central vertical axis and the central horizontal axis, wherein the first segment is the most distal segment of the implant, the second segment is the second-most distal segment of the implant, the third segment is the second-most proximal segment of the implant, and the fourth segment is the most proximal segment of the implant; wherein a maximum-height longitudinal cross-sectional area can be defined for each of the four segments, wherein each longitudinal cross-sectional area is parallel to the plane containing the central longitudinal axis and the central vertical axis, and wherein the maximum-height longitudinal cross-sectional area for a segment is that longitudinal cross-sectional area which contains the maximum distance between the lower surface and upper surface as measured along a vector that is parallel to the central vertical axis; wherein an upper perimeter can be defined for each of the four segments, wherein the upper perimeter is the upper portion of the maximum-height longitudinal cross-sectional area that is between the lateral cross-sectional areas that separate segments, wherein a lower perimeter can be defined for each of the four segments, wherein the lower perimeter is the lower portion of the maximum-height longitudinal cross-sectional area that is between the lateral cross-sectional areas that separate segments, wherein a segment maximum height can be defined for each segment, wherein the maximum height is the maximum distance between the segment's upper perimeter and lower perimeter as measured along a vector that is parallel to the central vertical axis; wherein a segment average height can be defined for each segment, wherein the average height is the average distance between the segment's upper perimeter and lower perimeter as measured along vectors that are parallel to the central vertical axis; wherein a segment upper slope can be defined as the slope of the straight line that best fits the segment's upper perimeter, wherein slope is defined as vertical change divided by longitudinal change when moving in a distal-to-proximal direction, and wherein the straight line that best fits the segment's perimeter is the straight line that minimizes the sum of squared deviations from the points comprising the perimeter; wherein a segment lower slope can be defined as the slope of the straight line that best fits the segment's lower perimeter, wherein slope is defined as vertical change divided by longitudinal change when moving in a distal-to-proximal direction, and wherein the straight line that best fits the segment's perimeter is the straight line that minimizes the sum of squared deviations from the points comprising the perimeter; wherein one or more of the conditions selected from the following group applies: the segment upper slope of segment three is more positive than the segment upper slope of segment two; and the segment lower slope of segment three is more negative than the segment lower slope of segment two; and wherein the segment average height of segment four is no less than the segment maximum height of segment three.

FIGS. 1 through 19 also show examples of how this invention can be embodied in a device wherein one or more of the conditions selected from the following group applies: the segment upper slope of segment three is at least 25% more positive than the segment upper slope of segment two; the segment lower slope of segment three is at least 25% more negative than the segment lower slope of segment two; the segment upper slope of segment four is at least 25% more positive than the segment upper slope of segment two; and the segment lower slope of segment four is at least 25% more negative than the segment lower slope of segment two.

FIGS. 1 through 19 show examples of how this invention can be embodied in a method for fusing spinal vertebrae comprising: (1) drilling a recess into a section of the spine comprising two spinal vertebrae; wherein this recess includes a portion of the intervertebral disk space, a portion of the upper vertebrae that is contiguous the intervertebral disk space, and a portion of the lower vertebrae that is contiguous the intervertebral disk space; wherein this recess extends between 25% and 75% of the lateral span of the intervertebral disk space; and wherein this recess is shaped like a section of a cone or rotated polygon; and wherein this recess has a wider proximal cross-section than distal cross-section; and (2) inserting an intervertebral implant into the intervertebral disk space and recess such that the distal end of the implant is substantially flush with the surface of the vertebrae on the side of the spinal column opposite the recess and the proximal end of the implant is substantially flush with the pre-drilling surface of the vertebrae on the side of the spinal column that has the recess. In an example, the proximal surface of the intervertebral implant substantially can conform to the wall of the recess when the intervertebral implant is inserted into the intervertebral space.

We claim:

1. An intervertebral implant for fusing spinal vertebrae comprising:
an implant that is implanted into the intervertebral disk space between two spinal vertebrae, wherein the following specifications apply to the implant excluding any fastening members which can be rotated or slid inwards independently of the implant;
the implant further comprising a distal portion that is first inserted into the intervertebral disk space, wherein this distal portion has a rounded distal end, two lateral surfaces, an upper surface, and a lower surface, wherein the best-fitting flat plane for the upper surface and the best-fitting flat plane for the lower surface are substantially parallel to each other, wherein the best-fitting flat plane for a surface is the flat plane that minimizes the sum or squared deviations from points on the surface; and wherein this distal portion spans at least 25% and no more than 75% of the distal-to-proximal length of the implant; and
the implant further comprising a proximal portion, wherein this proximal portion has an upper surface and a lower surface, wherein the best-fitting flat plane for the upper surface and the best-fitting flat plane for the lower surface are further apart at the proximal end of the proximal portion than they are at the distal end of the proximal portion, wherein the best-fitting flat plane for a surface is the flat plane that minimizes the sum or squared deviations from points on the surface, and wherein this proximal portion spans the remaining length of the distal-to-proximal length after accounting for the distal portion.

2. The device in claim 1 wherein the distal portion spans between 25% and 50% of the distal-to-proximal length of the implant and the proximal portion spans the remaining portion of the distal-to-proximal length of the implant.

3. The device in claim 1 wherein the distal portion spans between 50% and 75% of the distal-to-proximal length of the implant and the proximal portion spans the remaining portion of the distal-to-proximal length of the implant.

4. The device in claim 1 wherein the distal portion is shaped substantially like a rectangular column with substantially parallel upper and lower surfaces, with the possible exception of having rounded edges and a plurality of ridges or other protrusions on its upper and lower surfaces.

5. The device in claim 1 wherein the distal portion is shaped substantially like an elliptical column with a plurality of ridges or other protrusions on its upper and lower surfaces.

6. The device in claim 1 wherein the proximal portion is shaped substantially like a section of a cone that has a circular base and straight sides from the cone base to the peak.

7. The device in claim 1 wherein the proximal portion is shaped substantially like a section of a cone that has a circular base and convex sides from the cone base to the peak.

8. The device in claim 1 wherein the proximal portion is shaped substantially like a section of a cone that has a circular base and concave sides from the cone base to the peak.

9. The device in claim 1 wherein the proximal portion is shaped substantially like a section of a cone that has a elliptical base and straight sides from the cone base to the peak.

10. The device in claim 1 wherein the proximal portion is shaped substantially like a section of a cone that has a elliptical base and convex sides from the cone base to the peak.

11. The device in claim 1 wherein the proximal portion is shaped substantially like a section of a cone that has a elliptical base and concave sides from the cone base to the peak.

12. The device in claim 1 wherein the proximal portion is shaped substantially like a section of a rotated polygon.

13. The device in claim 1 wherein the proximal portion is shaped substantially like a section of a sphere.

14. The device in claim 1 wherein there are a plurality of ridges or other protrusions on the upper surface of the implant and/or on the lower surface of the implant in order to promote bone ingrowth and/or attachment of the implant to the vertebrae.

15. The device in claim 1 wherein there are a plurality of holes in the upper surface of the implant, in the lower surface of the implant, or extending from the upper surface of the implant to the lower surface of the implant in order to promote bone ingrowth, attachment of the implant to the vertebrae, and/or complete fusion of the vertebrae to each other.

16. An intervertebral implant for fusing spinal vertebrae comprising:
an implant that is implanted into the intervertebral disk space between two spinal vertebrae, wherein the following specifications apply to the implant excluding any fastening members which can be rotated and/or inserted inwards independently of the implant;
wherein the implant comprises a distal end, a proximal end, an upper surface, a lower surface, and two lateral surfaces, and wherein the distal end is the end that is first implanted into the intervertebral disk space;
wherein a central longitudinal axis can be defined for this implant, wherein this central longitudinal axis spans the implant from the distal end to the proximal end, wherein this central longitudinal axis is centrally located between the upper surface and the lower surface, wherein this central longitudinal axis is centrally located between the two lateral surfaces, and wherein this central longitudinal axis spans the maximum distance between the distal end and proximal end including any space that is fully or partially enclosed by the walls of the implant;
wherein a central vertical axis can be defined for this implant, wherein this central vertical axis spans the implant from the lower surface to the top surface, wherein this central vertical axis is perpendicular to the central longitudinal axis, wherein this central vertical axis is centrally located between the distal end and the proximal end, and wherein this central vertical axis is centrally located between the two lateral surfaces;
wherein a central horizontal axis can be defined for this implant, wherein this central horizontal axis spans the implant from one lateral side to the other lateral side, wherein this central horizontal axis is perpendicular to the central longitudinal axis, wherein this central horizontal axis is perpendicular to the central vertical axis, wherein this central horizontal axis is centrally located between the distal end and the proximal end, and wherein this central horizontal axis is centrally located between the lower surface and the upper surface;
wherein the implant can be longitudinally divided into four segments, wherein the length of the central longitudinal axis is divided into four equal linear portions, wherein there are three lateral cross-sectional areas separating these four equal linear portions, wherein each lateral cross-sectional area is parallel to the plane containing the central vertical axis and the central horizontal axis, wherein the first segment is the most distal segment of the implant, the second segment is the second-most distal segment of the implant, the third segment is the second-most proximal segment of the implant, and the fourth segment is the most proximal segment of the implant;
wherein a maximum-height longitudinal cross-sectional area can be defined for each of the four segments, wherein each longitudinal cross-sectional area is parallel to the plane containing the central longitudinal axis and the central vertical axis, and wherein the maximum-height longitudinal cross-sectional area for a segment is that longitudinal cross-sectional area which contains the maximum distance between the lower surface and upper surface as measured along a vector that is parallel to the central vertical axis;
wherein an upper perimeter can be defined for each of the four segments, wherein the upper perimeter is the upper portion of the maximum-height longitudinal cross-sectional area that is between the lateral cross-sectional areas that separate segments,
wherein a lower perimeter can be defined for each of the four segments, wherein the lower perimeter is the lower portion of the maximum-height longitudinal cross-sectional area that is between the lateral cross-sectional areas that separate segments,
wherein a segment maximum height can be defined for each segment, wherein the maximum height is the maximum distance between the segment's upper perimeter and lower perimeter as measured along a vector that is parallel to the central vertical axis;
wherein a segment average height can be defined for each segment, wherein the average height is the average distance between the segment's upper perimeter and lower perimeter as measured along vectors that are parallel to the central vertical axis;
wherein a segment upper slope can be defined as the slope of the straight line that best fits the segment's upper perimeter, wherein slope is defined as vertical change divided by longitudinal change when moving in a distal-to-proximal direction, and wherein the straight line that best fits the segment's perimeter is the straight line that minimizes the sum of squared deviations from the points comprising the perimeter;
wherein a segment lower slope can be defined as the slope of the straight line that best fits the segment's lower perimeter, wherein slope is defined as vertical change divided by longitudinal change when moving in a distal-to-proximal direction, and wherein the straight line that best fits the segment's perimeter is the straight line that minimizes the sum of squared deviations from the points comprising the perimeter;
wherein one or more of the conditions selected from the following group applies: the segment upper slope of segment three is more positive than the segment upper slope of segment two; and the segment lower slope of segment three is more negative than the segment lower slope of segment two; and
wherein the segment average height of segment four is no less than the segment maximum height of segment three.

17. The device in claim 16 wherein one or more of the conditions selected from the following group applies: the segment upper slope of segment three is at least 25% more positive than the segment upper slope of segment two; the segment lower slope of segment three is at least 25% more negative than the segment lower slope of segment two; the segment upper slope of segment four is at least 25% more positive than the segment upper slope of segment two; and the segment lower slope of segment four is at least 25% more negative than the segment lower slope of segment two.

18. The device in claim 16 wherein the distal portion is shaped substantially like a trapezoidal column, with the possible exception of having rounded edges and a plurality of ridges or other protrusions.

19. A method for fusing spinal vertebrae comprising:
drilling a recess into a section of the spine comprising two spinal vertebrae; wherein this recess includes a portion of the intervertebral disk space, a portion of the upper vertebrae that is contiguous the intervertebral disk space, and a portion of the lower vertebrae that is contiguous the intervertebral disk space; wherein this recess extends between 25% and 75% of the lateral span of the intervertebral disk space; and wherein this recess is shaped like a section of a cone or rotated polygon; and wherein this recess has a wider proximal cross-section than distal cross-section; and
inserting an intervertebral implant into the intervertebral disk space and recess such that the distal end of the implant is substantially flush with the surface of the vertebrae on the side of the spinal column opposite the recess and the proximal end of the implant is substantially flush with the pre-drilling surface of the vertebrae on the side of the spinal column that has the recess.

20. The method in claim 19 wherein the proximal surface of the intervertebral implant substantially conforms to the wall of the recess when the intervertebral implant is inserted into the intervertebral space.
